(19) United States
(12) Patent Application Publication
(10) Pub. No.: US 2007/0105214 A1
(43) Pub. Date: May 10, 2007

(54) Automated cellular assaying systems and related components and methods

(75) Inventors: Kenneth J. Micklash II, Royal Oak, MI (US); Jiyong Hong, Chapel Hill, NC (US); James Kevin Mainquist, San Diego, CA (US); Sheng Ding, San Diego, CA (US); Robert Charles Downs, La Jolla, CA (US); Peter G. Schultz, La Jolla, CA (US)

Correspondence Address: QUINE INTELLECTUAL PROPERTY LAW GROUP, P.C., P O BOX 458, ALAMEDA, CA, 94501, US

(73) Assignees: IRM, LLC, Hamilton (BM); THE SCRIPPS RESEARCH INSTITUTE, La Jolla, CA (US)

(21) Appl. No.: 11/271,501

(22) Filed: Nov. 9, 2005

(51) Int. Cl. C12M 3/08; B01L 3/00; C12M 1/34

(52) U.S. Cl. 435/306.1; 435/286.2; 435/287.3; 422/63; 422/99

(57) ABSTRACT

High throughput and automated cellular disruption systems that substantially uniformly disrupt cells as part of cellular motility assays as wells as other assay types are provided. In addition, various system components, including holding blocks, holding block loading devices, and system software, are also provided. Moreover, related methods that utilize these systems and system components are additionally provided.

### COPYRIGHT NOTIFICATION

Pursuant to 37 C.F.R. § 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The present invention relates generally to cellular assaying systems in addition to system components and associated methods.

### BACKGROUND OF THE INVENTION

Cell migration is a fundamental biological process, necessary for the spatial distribution of developing cell types and tissues, wound healing, blood vessel development, immune responses and renewal of cell layers in tissues such as the skin, esophagus and colorectum (Lauffenburger et al. (1996) Cell 84:359-369 and Ridley et al. (2003) Science 302:1704-1709, which are both incorporated by reference). The movements that constitute cell migration are complex, requiring the integration and transduction of diverse signaling cues with the mechanical processes of cell movement (Id.). Enhanced migration of tumor cells stems from the requirement to dissolve cell-cell contacts typical of organized epithelial structures, coupled with the acquisition of a mesenchymal phenotype (termed the epithelial-mesenchymal transition, EMT), which renders cells motile and invasive, resulting in enhanced extracellular matrix degradation and invasion, intra- and extravasion of blood vessels and, ultimately, distant metastases (Savagner et al. (2001) Bioessays 23:912-923, Thiery (2002) Nat Rev Cancer 2:442-454, Thiery et al. (2003) Curr Ovin Cell Biol 15:740-746, and Gotzmann et al. (2004) Mutat Res 566:9-20, which are each incorporated by reference).

Many key regulators of cell migration have been elucidated in different cell types and model organisms, including motility-associated extracellular matrix components and growth factors, the signal transduction networks that mediate these extracellular and integrin-sensed signals, and the mechanical effectors that mediate cell polarization, protrusion and adhesion formation, and retraction (Li et al. (2005) Annu Rev Biomed Eng 7:105-150, which is incorporated by reference). Although many of these molecular cues and signal cascades are active in cancer cells, a global view is lacking as to how cancer cells acquire enhanced motility and how this relates to changes in cell adhesion, mechanical movement, morphology and invasive capacity, as well as the interrelationship of these genetic programs.

The development of high throughput functional genomics screening approaches that utilize, e.g., RNA interference (Aza-Blanc et al. (2003) Mol Cell 12:627-637, Berns et al. (2004) Nature 428:431-437, and Willingham et al. (2004) Oncogene 23:8392-8400, which are each incorporated by reference), cDNA transfection (Strausberg et al. (2002) Proc Natl Acad Sci USA 99:16899-16903, Chanda et al. (2003) Proc Natl Acad Sci USA 100, 12153-12158, Matsuda et al. (2003) Oncogene 22:3307-3318, and Huang et al. (2004) Proc Natl Acad Sci USA 101:3456-3461, which are each incorporated by reference), and small molecules (Ding et al. (2003) Proc Natl Acad Sci USA 100:7632-7637, Kau et al. (2003) Cancer Cell 4:463-476, and Yarrow et al. (2003) Combinatorial Chemistry & High Throughput Screening 6:279-286, which are each incorporated by reference) in cells, coupled with advances in high-content visualization of cellular phenotypes (Kittler et al. (2004) Nature 432:1036-1040 and Yarrow (2004) Bmc Biotechnology 4:21, which are both incorporated by reference), makes tenable the genome-wide interrogation of cancer-associated cell behavior, among many other cellular properties or phenotypes. Many pre-existing cell migratory analyses have involved the use of manually operated cellular disruption or “scratch” devices that typically disrupt cells, for example, with inadequate uniformity and throughput. This lack of uniformity limits the comparability and reproducibility of cell migratory assay results. Moreover, these throughput limitations oftentimes make many of these pre-existing devices unsuitable for performing modern functional genomics screens, which commonly involve libraries with many thousands of compounds.

In order to apply high throughput screening technologies to a classic model of cell migration, automated cellular disruption systems that precisely and uniformly disrupt cells would facilitate these screening processes. These and many other features of the present invention will be apparent upon complete review of the following disclosure.

### SUMMARY OF THE INVENTION

The present invention relates generally to cell biology and to cell migratory analyses. More specifically, the invention provides automated cellular disruption systems that are configured to uniformly disrupt cells in repeatable modes that facilitate the reproducibility of cellular migration assays. Many pre-existing cellular motility assays are performed, for example, using hand-held cellular disruption devices that lack sufficient precision necessary to achieve reliably reproducible or uniform cellular disruption patterns (e.g., scratches, wounds, etc.). This lack of precision frequently yields biased assay results, among other deleterious consequences. In certain embodiments, the cellular disruption systems of the invention are coupled with automated high-speed microscopy, which allows for the rapid assessment of a cell's ability to close a uniform wound, scratch, or other disruption in multi-well tissue culture plates. In addition to various system components (e.g., holding block loading devices, holding blocks, system software, etc.), the invention also provides related methods.

In one aspect, the invention provides an automated cellular disruption system. The system includes at least one cellular disruption component, at least one container positioning component (e.g., a container nest, etc.) structured to position at least one container, and at least one translational mechanism operably connected to the cellular disruption component and/or the container positioning component. In addition, the system also includes at least one controller operably connected to the translational mechanism. The controller is configured to direct the translational mechanism to move the cellular disruption component and/or the container positioning component relative to one another in at least one substantially uniform mode such that the cellular disruption component disrupts cells disposed in the container when the container positioning component positions the container. The substantially uniform mode typically comprises one or more selectable parameters selected from, e.g., a distance of cellular disruption component and/or container positioning component movement, a pathway of cellular disruption component and/or container positioning component movement, a rate of cellular disruption component and/or container positioning component movement, a level of force applied by the cellular disruption component and/or the container positioning component on the container, etc.

In another aspect, the invention provides an automated cellular disruption system. The system includes a cellular disruption component comprising multiple mechanical disruption devices. The system also includes a container positioning component (e.g., a container nest, etc.) structured to position a container, and a translational mechanism operably connected to the cellular disruption component and/or the container positioning component. In addition, the system also includes a controller operably connected to the translational mechanism. The controller is configured to direct the translational mechanism to move the cellular disruption component and/or the container positioning component relative to one another along a first axis such that at least two of the mechanical disruption devices contact at least one surface of the container comprising cells with substantially constant force when the container positioning component positions the container. In some embodiments, the substantially constant force causes the mechanical disruption devices to deflect away from the first axis (e.g., a Z-axis) when the mechanical disruption devices contact the surface of the container. In certain embodiments, the controller is configured to move the cellular disruption component and/or the container positioning component relative to one another along at least a second axis (e.g., an X- and/or Y-axis) when the container positioning component positions the container and the mechanical disruption devices are in contact with the surface of the container. In certain embodiments, the container positioning component is structured to position a multi-well container. In these embodiments, the multiple mechanical disruption devices are typically configured to correspond to at least a subset of wells of the multi-well container such that the multiple mechanical disruption devices contact surfaces of the wells of the multi-well container comprising the cells with the substantially constant force when the container positioning component positions the multi-well container.

In another aspect, the invention provides an automated cellular disruption system. The system includes at least one cellular disruption component comprising a holding block receiving area that is structured to receive a holding block that is structured to hold at least one cellular disruption implement. The system also includes at least one translational mechanism operably connected to the cellular disruption component. In addition, the system also includes at least one controller operably connected to the translational mechanism. The controller is configured to direct the translational mechanism to move the cellular disruption component such that the cellular disruption component disrupts cells disposed in at least one container when the holding block holds the cellular disruption implement, the holding block receiving area receives the holding block, and the container is positioned relative to the cellular disruption component. The controller is typically configured to direct the translational mechanism to move the cellular disruption component in at least one substantially uniform mode. In some embodiments, the system includes a container positioning component (e.g., a container nest, etc.) structured to position one or more containers. In these embodiments, the translational mechanism is typically operably connected to the container positioning component and the controller is configured to move the container positioning component and the cellular disruption component relative to one another. Typically, the holding block receiving area of the system includes the holding block. In some of these embodiments, the holding block holds the cellular disruption implement.

As referred to above, the automated cellular disruption systems described herein typically include translational mechanisms that move cellular disruption components and/or container positioning components relative to one another. In some embodiments, for example, translational mechanisms comprise linear actuators operably connected to cellular disruption components, e.g., to move those components along at least a first axis, such as a Z-axis. To further illustrate, translational mechanisms optionally include air tables operably connected to container positioning components, e.g., to move container positioning components along at least a second axis (e.g., an X- and/or Y-axis).

The cellular disruption components of the systems described herein include various embodiments. In some embodiments, for example, cellular disruption components comprise at least one cellular disruption implement selected from, e.g., a radiation source, an electrical source, a thermal source, a mechanical disruption device, and the like. Exemplary mechanical disruption devices include a pipette tip, a prong, a pin, a needle, a scraper, a razor, etc.

In some embodiments, the cellular disruption component of the systems described herein comprises a holding block receiving area that comprises a holding block that holds the cellular disruption implement (e.g., one or more mechanical disruption devices, etc.). Typically, the cellular disruption implement includes at least one locating feature structured to locate the cellular disruption implement relative to the holding block (e.g., along a Z-axis, etc.). In certain embodiments, the holding block receiving area comprises at least one actuating mechanism operably connected to at least one cellular disruption implement locating component. The actuating mechanism is generally configured to reversibly move the cellular disruption implement locating component such that the cellular disruption implement locating component applies a substantially constant force to the cellular disruption implement held by the holding block. In these embodiments, an elastomeric material is optionally disposed between the cellular disruption implement locating component and the holding block. In some embodiments, the cellular disruption implement locating component includes at least one top support and at least one bottom support operably connected to the actuating mechanism. In these embodiments, the holding block is typically structured to be positioned between the top and bottom supports. In some of these embodiments, at least one peg extends from the top support and is resiliently coupled to the top support by a resilient coupling (e.g., a spring or the like). In these embodiments, the peg is generally configured to contact the cellular disruption implement when the holding block is positioned in the holding block receiving area, e.g., to securely and compliantly position or locate the cellular disruption implement in the holding block. Furthermore, at least one surface of the container positioning component and at least one surface of the cellular disruption implement locating component are typically substantially parallel with one another, e.g., to effect precise positioning of the cellular disruption implement and a container relative to one another when the container is positioned on the container positioning component.

In some embodiments, the cellular disruption component comprises multiple cellular disruption implements. In these embodiments, the controller is typically configured to direct the translational mechanism to move the cellular disruption component and/or the container positioning component relative to one another such that the multiple cellular disruption implements substantially uniformly disrupt the cells disposed in the container when the container positioning component positions the container. In some of these embodiments, the container positioning component is structured to position a multi-well container. In these embodiments, the multiple cellular disruption implements are generally configured to correspond to at least a subset of wells of the multi-well container such that the multiple cellular disruption implements substantially uniformly disrupt the cells disposed in the wells of the multi-well container when the translational mechanism moves the cellular disruption component and/or the container positioning component relative to one another and the container positioning component positions the multi-well container.

As referred to above, the cellular disruption components of the systems described herein optionally comprise mechanical disruption devices in certain embodiments. In these embodiments, controllers are typically configured to direct translational mechanisms to move cellular disruption components and/or container positioning components along a first axis (e.g., a Z-axis) such that the mechanical disruption device deflects away from the first axis upon contacting the container when the container positioning component positions the container. Typically, controllers are configured to direct translational mechanisms to move cellular disruption components and/or container positioning components along the first axis such that the mechanical disruption devices apply a unit load sufficient to move at least portions of containers (e.g., the bottom walls of wells in a multi-well container, etc.) between about 0.20 mm and about 0.55 mm relative to initial positions of the portions of the containers. In some embodiments, controllers are configured to direct translational mechanisms to move cellular disruption components and/or container positioning components along at least a second axis (e.g., a X- and/or Y-axis) after the mechanical disruption devices contact the containers when the container positioning components position the containers.

The automated cellular disruption systems described herein optionally include one or more additional components. Examples of these additional components include: a robotic gripping component structured to grip and translocate containers between the container positioning component and another location; an assaying component structured to assay cells; a material handling component structured to dispense and/or remove material from one or more containers; an incubation component structured to incubate containers; a container storage component structured to store containers; and a detection component structured to detect detectable signals produced in containers. For example, the detection component optionally comprises an imaging device that is configured to capture one or more images of cells disposed in the containers.

In another aspect, the invention provides a holding block loading device that includes a support plate and a plurality of protrusions that protrude from a surface of the support plate. The protrusions are configured to substantially correspond to a plurality of orifices disposed through a holding block and structured to engage pipette tips. Typically, the protrusions are configured to correspond to at least a subset of wells of at least one multi-well container. In some embodiments, the protrusions are non-fluid conveying. The holding block loading device also includes a disengagement plate comprising a plurality of holes through which the plurality of protrusions protrude. The disengagement plate is structured to selectively move relative to the protrusions to disengage the pipette tips from the protrusions when the protrusions engage the pipette tips. In some embodiments, holding block loading device includes a resilient coupling that couples the support plate and the disengagement plate to one another, and/or a retaining mechanism structured to selectively retain the disengagement plate at least one position relative to the support plate.

In another aspect, the invention provides a holding block that includes a body structure that is structured to hold at least one cellular disruption implement and to be received by a holding block receiving area of an automated cellular disruption system. Typically, at least one orifice is disposed through the body structure. The orifice is structured to receive and retain the cellular disruption implement. In some embodiments, the cellular disruption implement extends from the body structure when the body structure holds the cellular disruption implement. In these embodiments, the body structure is typically structured to substantially limit deflection of the cellular disruption implement at regions other than those that extend from the body structure. Typically, the body structure is structured to hold multiple cellular disruption implements in a configuration that corresponds to at least a subset of wells of at least one multi-well container. Optionally, the holding block includes the cellular disruption implement. In these embodiments, the cellular disruption implement is typically selected from, e.g., a radiation source, an electrical source, a thermal source, a mechanical disruption device, etc. Furthermore, the mechanical disruption device is generally selected from, e.g., a pipette tip, a prong, a pin, a needle, a scraper, a razor, and the like.

In another aspect, the invention provides a computer program product that includes a computer readable medium that comprises one or more logic instructions for moving a cellular disruption component and/or a container positioning component of an automated cellular disruption system relative to one another such that the cellular disruption component disrupts cells disposed in a container positioned by the container positioning component. In certain embodiments, the computer readable medium comprises at least one logic instruction for receiving at least one input parameter selected from, e.g., a distance of cellular disruption component and/or container positioning component movement, a pathway of cellular disruption component and/or container positioning component movement, a rate of cellular disruption component and/or container positioning component movement, a level of force applied by the cellular disruption component and/or the container positioning component on the container, a container format, and the like. In some embodiments, the computer readable medium comprises at least one logic instruction for moving the cellular disruption component and/or the container positioning component along a first axis such that at least one cellular disruption implement of the cellular disruption component contacts at least one surface of the container comprising the cells when the container is positioned relative to the automated cellular disruption system, and moving the cellular disruption component and/or the container positioning component along at least a second axis to disrupt the cells when the container is positioned by the container positioning component and the cellular disruption implement contacts the surface of the container. In these embodiments, the computer readable medium optionally includes at least one logic instruction for contacting the cellular disruption implement with the surface of the container with sufficient force to deflect the cellular disruption implement away from the first axis.

In another aspect, the invention provides a method of disrupting cells. The method includes (a) providing an automated cellular disruption system that comprises at least one cellular disruption component, and (b) providing cells (e.g., mammalian cells, etc.) disposed on at least one surface of at least one container. The cells typically include normal cells, transformed cells, infected cells, cancer cells, and/or the like. The method also includes (c) moving the cellular disruption component and/or the container at least one selected distance in at least one substantially uniform mode such that the cellular disruption component disrupts the cells disposed on the surface of the container.

The method includes various embodiments. In some embodiments, for example, the method includes repeating (b) and (c) at least once using at least one other container. Typically, the method includes selecting the substantially uniform mode prior to (c) in which the substantially uniform mode comprises one or more selectable parameters selected from, e.g., a distance of cellular disruption component and/or container positioning component movement, a pathway of cellular disruption component and/or container positioning component movement, a rate of cellular disruption component and/or container positioning component movement, a level of force applied by the cellular disruption component and/or the container positioning component on the container, and the like. To further illustrate, the cellular disruption component optionally comprises at least one cellular disruption implement selected from, e.g., a radiation source, an electrical source, a thermal source, and a mechanical disruption device, and (c) includes photobleaching the cells, applying an electric field to the cells, laser ablating the cells, applying thermal energy to the cells, exposing the cells to ultra-violet radiation, mechanically disrupting the cells, and/or the like.

In certain embodiments, the container includes a multi-well container having the cells disposed in wells thereof, and the automated cellular disruption system comprises multiple cellular disruption implements that are configured to correspond to at least a subset of wells of the multi-well container. In these embodiments, (c) comprises substantially uniformally disrupting the cells in at least the subset of the wells of the multi-well container in parallel. Optionally, the multiple cellular disruption implements comprise multiple mechanical disruption devices, and (c) comprises moving the cellular disruption component and/or the multi-well container along a first axis (e.g., a Z-axis) such that the mechanical disruption devices deflect away from the first axis under a substantially constant applied force upon contacting the multi-well container, and moving the cellular disruption component and/or the multi-well container along at least a second axis (e.g., an X- and/or Y-axis) to disrupt the cells in the wells of the multi-well container in parallel.

To further illustrate, the cellular disruption component optionally comprises a holding block receiving area that is structured to receive a holding block that is structured to hold at least one cellular disruption implement. In these embodiments, the method generally comprises positioning the cellular disruption implement such that the holding block holds the cellular disruption implement and positioning the holding block in the holding block receiving area prior to (c). In some embodiments, for example, the cellular disruption implement comprises a pipette tip, and the method comprises positioning the pipette tip using a holding block loading device.

Typically, the method includes contacting the cells with, or introducing into the cells, at least one modulator or at least one candidate modulator prior to, during, and/or after (b). For example, the method optionally comprises a cell motility assay and/or a cell viability assay. Exemplary modulators or candidate modulators include an inorganic molecule, an organic molecule, a vector comprising or encoding the modulator or the candidate modulator, a sense nucleic acid, an anti-sense nucleic acid, a transcription factor, a complementary DNA (cDNA), an short interfering RNA (siRNA), a microRNA (miRNA), a synthetic hairpin RNA (shRNA), and the like.

In some embodiments, the method includes detecting at least one detectable property of the cells prior to, during, and/or after (b). For example, this optionally includes imaging the cells prior to, during, and/or after (b). The detectable property typically comprises a presence or absence of cellular motility. The method typically includes correlating the detected detectable property with at least one gene of the cells, and/or comparing the detected detectable property with at least one control.

In certain embodiments, the automated cellular disruption system comprises at least one container positioning component structured to position the container. In these embodiments, the method generally comprises positioning the container on the container positioning component prior to (c). Optionally, the automated cellular disruption system comprises at least one translational mechanism operably connected to the cellular disruption component and/or the container positioning component, and at least one controller operably connected to the translational mechanism. In these embodiments, (c) typically includes moving the cellular disruption component and/or the container positioning component relative to one another.

### BRIEF DESCRIPTION OF THE DRAWINGS

FIG. 1 schematically shows an automated cellular disruption system from a perspective view according to one embodiment of the invention.

FIG. 2A schematically illustrates a cellular disruption component in an open position from a perspective view.

FIG. 2B schematically depicts the cellular disruption component of FIG. 2A in a closed position from a front elevational view.

FIG. 2C schematically shows the cellular disruption component of FIG. 2A along with a container positioning component from a perspective view.

FIG. 2D schematically illustrates a cellular disruption component that includes resiliently coupled pegs positioning cellular disruption implements in a holding block from a perspective view according to one embodiment of the invention.

FIG. 3A schematically shows a holding block from a transparent side elevational view according to one embodiment of the invention.

FIG. 3B schematically depicts the holding block of FIG. 3A from a transparent top view.

FIG. 3C schematically illustrates the holding block of FIG. 3A from a transparent perspective view.

FIG. 4A schematically shows a holding block loading device positioned over a pipette tip box according to one embodiment of the invention.

FIG. 4B schematically depicts the protrusions of a holding block loading device engaging pipette tips in a pipette tip box according to one embodiment of the invention.

FIG. 4C schematically illustrates the protrusions of a holding block loading device engaging pipette tips according to one embodiment of the invention.

FIG. 4D schematically shows a holding block engaging pipette tips loaded on the protrusions of a holding block loading device according to one embodiment of the invention.

FIG. 4E schematically depicts a user disengaging the protrusions of a holding block loading device from pipette tips according to one embodiment of the invention.

FIG. 4F schematically shows pipette tips positioned in a holding block according to one embodiment of the invention.

FIG. 5A schematically shows a pipette tip from a side elevational view.

FIG. 5B schematically illustrates a prong from a side elevational view.

FIG. 5C schematically depicts a needle from a side elevational view.

FIG. 5D schematically depicts a scraper from a side elevational view.

FIG. 5E schematically shows in a pin from a side elevational view.

FIG. 5F schematically illustrates a razor from a side elevational view.

FIG. 6A schematically shows a cellular disruption component that includes lasers as cellular disruption implements from a front elevational view according to one embodiment of the invention.

FIG. 6B schematically illustrates a cellular disruption component that includes electrodes as cellular disruption implements from a front elevational view according to one embodiment of the invention.

FIG. 7A schematically shows a container nest from a perspective according to one embodiment of the invention.

FIG. 7B schematically depicts the container nest of FIG. 7A from a side elevational view.

FIG. 8 schematically depicts a container positioning component from a top perspective view according to one embodiment of the invention.

FIG. 9A schematically shows a top view of a microtiter plate.

FIG. 9B schematically illustrates a bottom view of the microtiter plate shown in FIG. 9A.

FIG. 9C schematically depicts a cross-sectional view of the microtiter plate shown in FIG. 9A.

FIG. 10 schematically shows an assaying component from a perspective view according to one embodiment of the invention.

FIG. 11 schematically depicts one embodiment of a robotic gripping component from a side elevational view.

FIG. 12 schematically illustrates one embodiment of a grasping mechanism coupled to a boom of a robot from a perspective view.

FIG. 13A schematically illustrates another embodiment of a grasping mechanism coupled to a boom of a robot from a perspective view.

FIG. 13B schematically shows another exemplary embodiment of a grasping mechanism from a top perspective view.

FIG. 13C schematically depicts the grasping mechanism from FIG. 13B from a bottom perspective view.

FIG. 13D schematically shows a pivot member from a front elevational view according to one embodiment.

FIG. 13E schematically illustrates a pivot member from a front elevational view according to another embodiment.

FIG. 14A schematically shows a dispensing system from a perspective view according to one embodiment of the invention.

FIG. 14B schematically illustrates a detailed bottom perspective view of a dispensing component from the dispensing system of FIG. 14A.

FIG. 14C schematically depicts a detailed top perspective view of a dispensing component from the dispensing system of FIG. 14A.

FIG. 15A schematically depicts a front cutaway view of one embodiment of an incubation component.

FIG. 15B schematically depicts a side cutaway view of the incubation component shown in FIG. 15A.

FIG. 16A schematically depicts a top cutaway view of one embodiment of an incubation component.

FIG. 16B schematically depicts a bottom cutaway view of the incubation component shown in FIG. 16A.

FIG. 17A schematically depicts a front view of one embodiment of an incubation component.

FIG. 17B schematically depicts a top view of the incubation component shown in FIG. 17A.

FIG. 18 schematically depicts a robotic gripping component interfacing with a door of an incubation component from a perspective view.

FIG. 19 schematically illustrates a modular object storage component and a robotic gripping component from a perspective view.

FIG. 20A are captured images that show the temporal (0, 4, 8, 12 and 16 hrs) migration of SKOV-3 cells in the presence and absence of controls, where siRNA-con=FITC-conjugated control siRNA; siRNA−RAC=a sequence-specific siRNA targeting RAC; DMSO=dimethyl sulfoxide; and SA1001=c-src family kinase inhibitor, compound 43 (Goldberg et al. (2003) J. Med. Chem. 46:1337-1349, which is incorporated by reference).

FIG. 20B are photographs of SDS-PAGE/Western blots that demonstrate the knock-down of the RAC protein by the RAC-specific siRNA used in the analysis described with respect to FIG. 20A, compared to a control siRNA (CON) and mock transfected cells (LIPO). Photographs of the same blot re-probed with anti-actin antibody to demonstrate equal loading are also shown FIG. 20B.

FIG. 21 schematically depicts a SKOV-3 siRNA screen and the associated follow-up.

FIG. 22 shows identification and validation of pro-migratory genes by phenotypic and transcriptional analysis. The migratory inhibition elicited by two independent siRNA duplexes targeting four genes, MAP4K4, CDK7, DYRK1B and SERPINB3, is shown compared to control siRNA and quantified by the automated algorithm (black bars=migration score; white bars=relative cellular viability). RT-PCR analysis is shown for each transcript, and the relative transcriptional knockdown was quantified using ImageJ software (downloaded from the NIH website).

FIG. 23 schematically illustrates a representative system in which various aspects of the present invention may be embodied.

### DETAILED DESCRIPTION

### I. DEFINITIONS

Before describing the present invention in detail, it is to be understood that this invention is not limited to particular embodiments. It is also to be understood that the terminology used herein is for the purpose of describing particular embodiments only, and is not intended to be limiting. As used in this specification and the appended claims, the singular forms “a,” “an,” and “the” also include plural referents unless the context clearly provides otherwise. Thus, for example, reference to “a cellular disruption implement” also includes more than one cellular disruption implement. Units, prefixes, and symbols are denoted in the forms suggested by the International System of Units (SI), unless specified otherwise. Numeric ranges are inclusive of the numbers defining the range. Further, unless defined otherwise, all technical and scientific terms used herein have the same meaning as commonly understood by one of ordinary skill in the art to which the invention pertains. The terms defined below, and grammatical variants thereof, are more fully defined by reference to the specification in its entirety.

The term “automated” refers to a process, device, sub-system, or system that is controlled at least partially by mechanical and/or electronic devices in lieu of direct human control. In certain embodiments, for example, the systems of the invention are configured to disrupt cells disposed on container surfaces in the absence of direct human control.

The term “bottom” refers to the lowest point, level, surface, or part of a system, device, or component thereof, when oriented for typical designed or intended operational use.

Objects “correspond” to one another when the objects, or component parts thereof, can interact with one another. In some embodiments, for example, multiple cellular disruption implements are configured or arranged such that individual cellular disruption implements can concurrently contact the bottom surfaces or walls of different wells in a given multi-well container. To further illustrate, the protrusions of a holding block loading device typically include a plurality of protrusions that are configured to be inserted into a plurality of orifices disposed through a holding block.

The term “disrupt” in the context of a cellular migration assay or the like refers to an interruption or disturbance of a confluent cell population or of another course, pattern, or unity of cellular growth in a cell culture container. In certain assays, for example, contact between some cells in confluent cellular monolayers is interrupted or disturbed by “scratching” (e.g., physically moving cells from portions of) surfaces of containers that include the cells.

The term “substantially” refers to an approximation. In certain embodiments, for example, mechanical disruption devices contact container surfaces under a constant or approximately constant applied force.

A “system” refers a group of objects and/or devices that form a network for performing a desired objective. In some embodiments, for example, a system of the invention includes a translational mechanism operably connected to a cellular disruption component and a container positioning component such that those components move relative to one another to effect the disruption of cells disposed in a container positioned on the container positioning component.

The term “top” refers to the highest point, level, surface, or part of a system, device, or component thereof, when oriented for typical designed or intended operational use.

The term “uniform mode” refers to a repeatable form or arrangement of something. In some embodiments, for example, system controllers are configured to direct translational mechanisms to move cellular disruption components and/or container positioning components in repeatable arrangements. Uniform modes typically include one or more unvarying or constant parameters, such as a level of force applied by the cellular disruption component and/or the container positioning component on the container, a distance, pathway, and/or rate of cellular disruption component and/or container positioning component movement, and the like.

### II. INTRODUCTION

While the present invention will be described with reference to a few specific embodiments, the description is illustrative of the invention and is not to be construed as limiting the invention. As will be apparent to those skilled in the art to which this invention pertains, various modifications can be made to certain embodiments of the invention without departing from the true scope of the invention as defined by the appended claims. It is noted here that for a better understanding, like components are designated by like reference letters and/or numerals throughout the various figures.

Cell motility is a complex biological process, integral to normal development, tissue remodeling, immunity, and angiogenesis. In diseases such as cancer, particularly those arising in highly organized epithelial tissues, the acquisition of a migratory phenotype is a critical step toward tissue invasion and metastatic spread. The present invention relates to genetic screens that identify components of cancer-associated cell migration as well as other disease states using precision engineered cellular disruption or wound healing systems, which are typically coupled with automated microscopy systems. The systems described herein generally achieve much higher throughput along with greater wound uniformity and reproducibility than many pre-existing devices, which are typically manually operated. An example that involved a highly motile ovarian carcinoma cell line screened across an arrayed short interfering RNA (siRNA) library using a representative cellular disruption system of the invention is provided below.

Aside from automated cellular disruption systems, various system components, such as holding blocks, holding block loading devices, and system software, are also provided. In addition, the invention also provides related methods that utilize these systems and system components. Each of theses aspects of the invention as well as others are described in greater detail below.

### III. AUTOMATED CELLULAR DISRUPTION SYSTEMS AND SYSTEM COMPONENTS

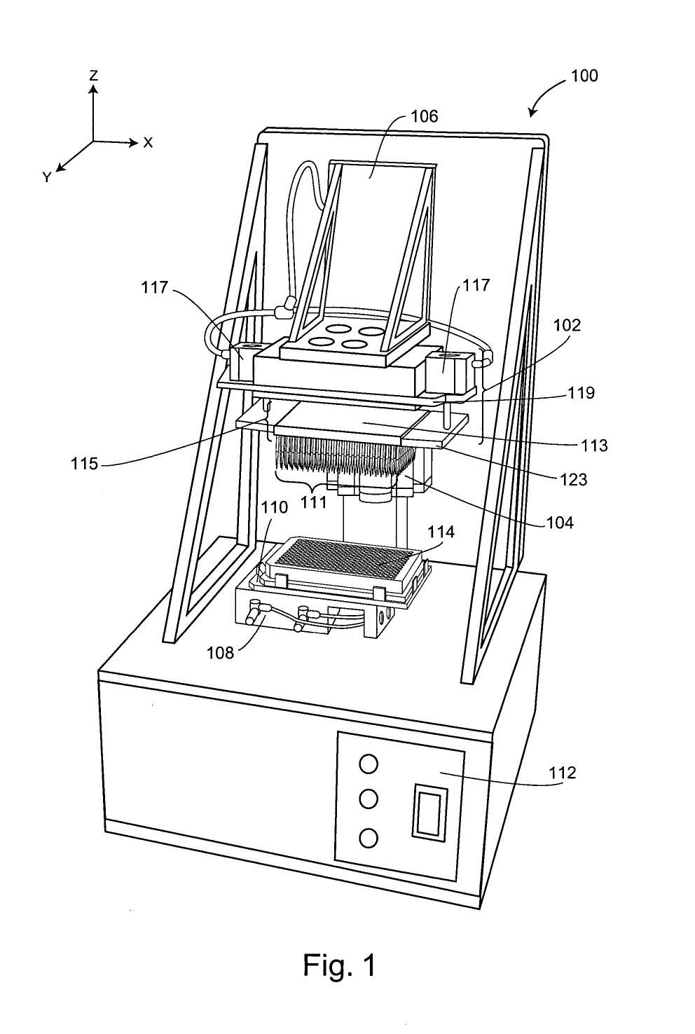

The present invention provides automated cellular disruption systems in addition to various system components. Referring initially to FIG. 1, automated cellular disruption system 100 is schematically shown from a perspective view according to one embodiment of the invention. As shown, automated cellular disruption system 100 includes cellular disruption component 102 operably connected to translational mechanism 104 (shown as a linear motion component comprising, e.g., a linear actuator) via mounting bracket 106. As also shown, cellular disruption component 102 includes an array of cellular disruption implements 111 (shown as pipette tips) held within holding block 113, which is disposed in holding block receiving area 115. Translational mechanism 108 (shown as an air table) is operably connected to container positioning component 110 (shown as a container nest). Automated cellular disruption system 100 also includes controller 112, which is operably connected to cellular disruption component 102 and translational mechanisms 104 and 108. Controller 112 is configured to direct cellular disruption component 102 to move between open and closed positions. Controller 112 is also configured to direct translational mechanism 104 to move cellular disruption component 102 along the Z-axis and translational mechanism 108 to move container positioning component 110 along the X-axis to effect the disruption of cells disposed in the wells of multi-well container 114 (shown as a 384-well microtiter plate corresponding to cellular disruption implements 111). Each of these system components is described further below.

A. Cellular Disruption Components

There are a variety of cellular disruption components that can be utilized, or adapted for use, in the systems described herein to effect the disruption of cell populations, e.g., as part of cellular motility assays. In some embodiments, for example, cellular disruption components include holding block receiving areas that are structured to receive and precisely position removable holding blocks. Holding blocks, which are described further below, are fabricated to hold cellular disruption implements, such as mechanical disruption devices or other types of implements. In other exemplary embodiments, cellular disruption implements are manufactured as integral parts of cellular disruption components (e.g., cellular disruption components lack holding block receiving areas).

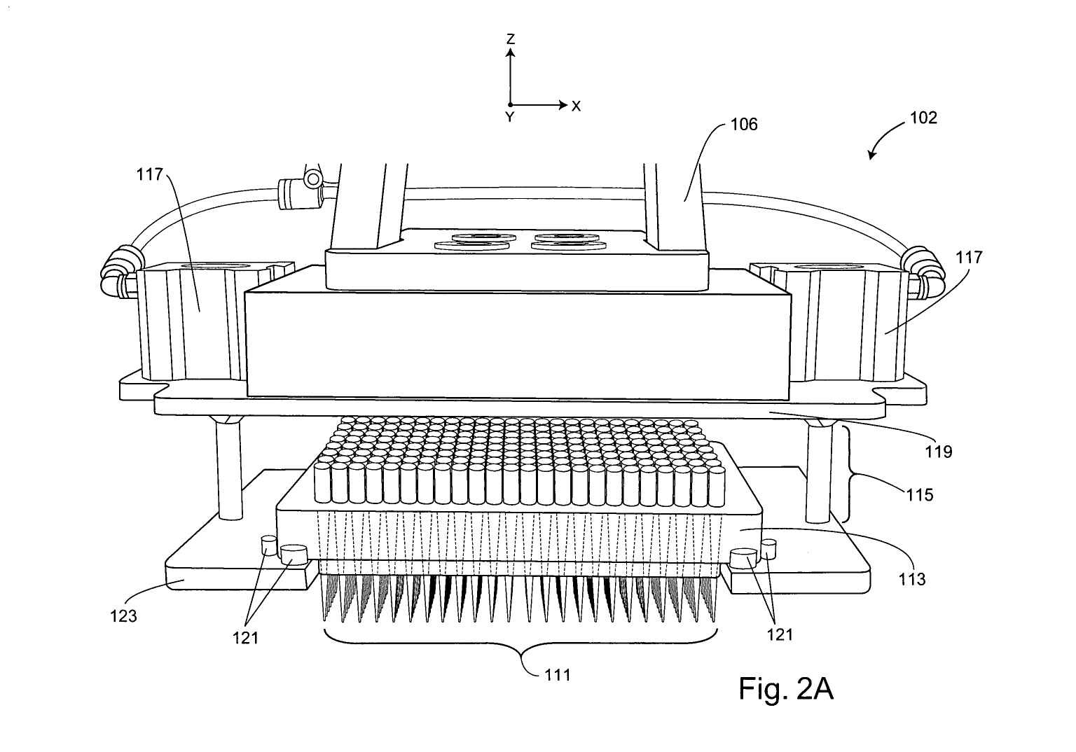

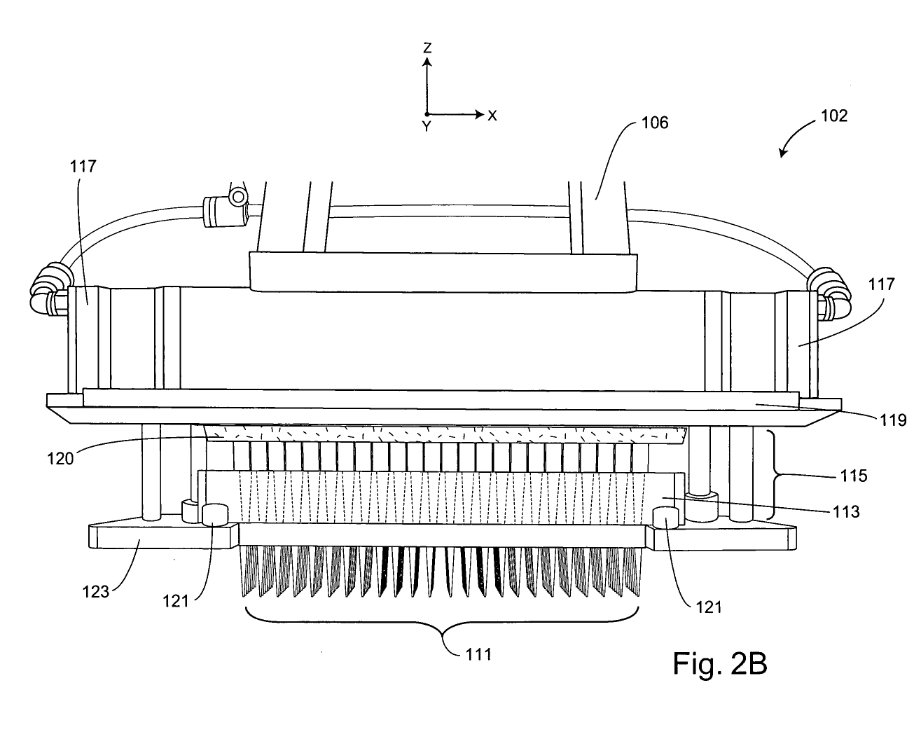

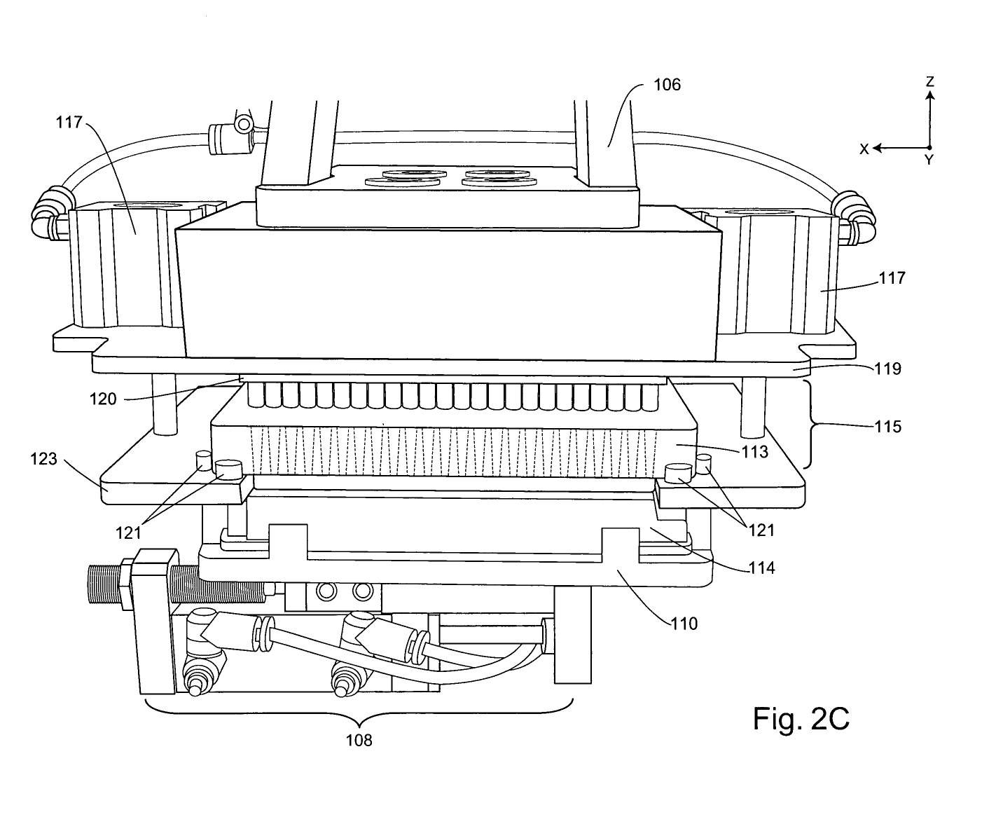

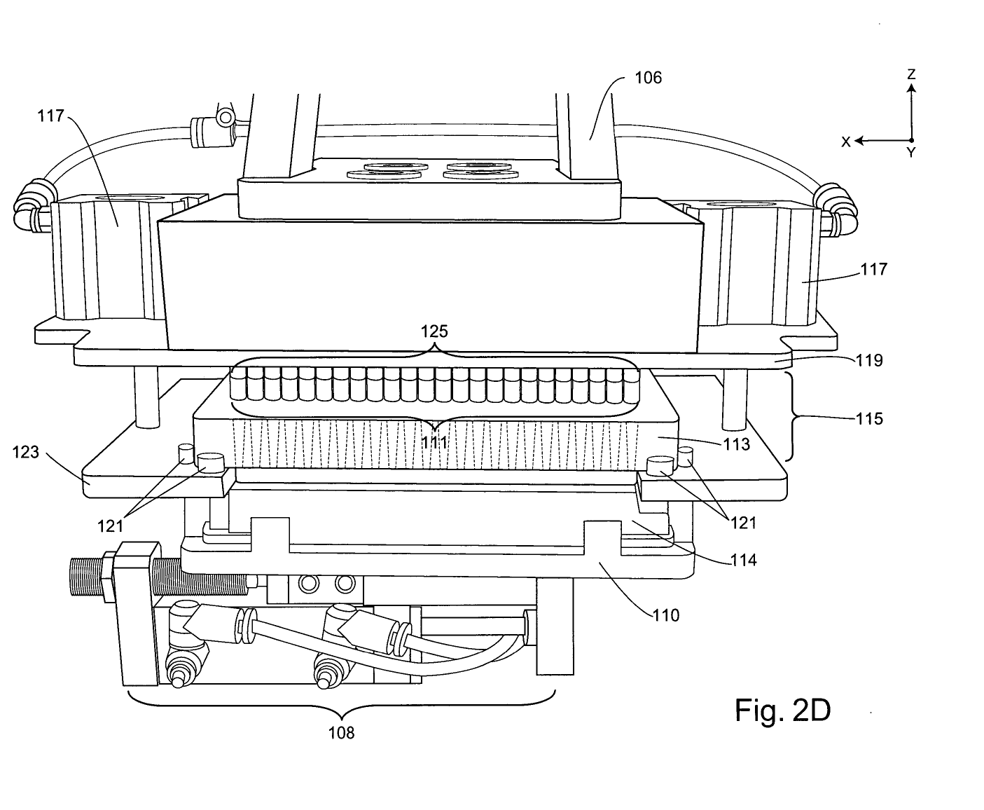

To further illustrate, FIGS. 2A-C schematically show detailed views of cellular disruption component 102 of automated cellular disruption system 100. In particular, FIG. 2A schematically illustrates cellular disruption component 102 in an open position from a perspective view. As shown, holding block receiving area 115 is formed by top support 119 and bottom support 123 (each shown as a plate). During operation, holding block 113 is typically positioned on bottom support 123 with cellular disruption component 102 in the open position. In the embodiment depicted, bottom support 123 includes alignment features 121, which are structured to align holding block 113 relative to bottom support 123. Cellular disruption component 102 also includes actuating mechanisms 117 (shown as air cylinders), which reversibly move bottom support 123 relative to top support 119 along the Z-axis to open and close cellular disruption component 102.

Once holding block 113 is positioned on bottom support 123, as shown in FIG. 2A, elastomeric material 120 (shown in FIG. 2B as a gasketing sheet) is typically placed between holding block 113 and top support 119 in holding block receiving area 115. Elastomeric material 120 assists in securely locating cellular disruption implements 111 and holding block 113 relative to one another and to cellular disruption component 102 when cellular disruption component 102 is in a closed position (see, FIG. 2B). In addition to securely locating cellular disruption implements 111, elastomeric material 120 also provides a certain degree of compliance to cellular disruption implements 111 positioned in holding block 113 depending upon the particular elastomeric material that is used in a given application. In certain embodiments, elastomeric materials are adhered or otherwise attached to top supports, whereas in other embodiments, elastomeric materials or functional equivalents are omitted. In some embodiments, other components, such as pegs or the like are used in lieu of, or in addition to, elastomeric materials to locate cellular disruption implements in the systems described herein. For example, FIG. 2D schematically illustrates pegs 125, which are each individually coupled to top support 119 by a resilient coupling, such as a spring, etc. As shown, pegs 125 contact cellular disruption implements 111 when holding block receiving area 115 is in a closed position. Optionally, pegs 125 are coupled to top support 119 in fixed positions, e.g., in the absence of resilient couplings.

Essentially any elastomeric or gasketing material is optionally utilized to securely locate cellular disruption implements and holding blocks relative to one another and to cellular disruption components. For example, suitable gasket sheets are optionally fabricated from, e.g., foam rubber, VITON®, SANTOPRENE®, TEFLON®, GORE-TEX®, Celerus™, or the like. Many of these materials are readily available from various commercial suppliers, such as W. L. Gore & Associates (Newark, Del., USA). Combinations of materials, e.g., in the form of laminates are also optionally utilized as gasketing sheets in the systems of the invention.

As shown in FIG. 2B, once elastomeric material 120 is placed between holding block 113 and top support 119, actuating mechanisms 117 are typically activated to move cellular disruption component 102 into a closed position in which top support 119, applies a substantially constant force to cellular disruption implements 111 held by holding block 113. Cellular disruption implements are described further below. Top support 119 and bottom support 123 together function as a cellular disruption implement locating component when cellular disruption component 102 is in the closed position by securely, precisely, and compliantly positioning cellular disruption implements 111 relative to one another in holding block 113. Thereafter, translational mechanism 104 typically lowers cellular disruption component 102 along the Z-axis until cellular disruption implements 111 contact the bottom surfaces of wells disposed in multi-well container 114, which is positioned on container positioning component 110 (see, FIG. 2C). As shown in FIG. 2C, the horizontal surfaces of top support 119, bottom support 123, and container positioning component 110 are substantially parallel with one another so that cellular disruption implements 111 uniformly contact the bottom surfaces of wells disposed in multi-well container 114 during this process. The bottom surfaces of these wells typically comprise populations of cells (e.g., confluent monolayers of cells). In some embodiments, translational mechanism 108 is then engaged to move container positioning component 110 along the X-axis a selected distance such that the cells disposed on the bottom surfaces of the wells of multi-well container 114 are substantially uniformly disrupted (i.e., the wounds or “scratches” generated are substantially the same in each well of multi-well container 114).

In other embodiments, the automated cellular disruption systems of the invention are configured to disrupt cells in substantially uniform modes, and/or with cellular disruption implements, that differ from those described above with respect to FIGS. 2A-C. Additional exemplary substantially uniform modes and cellular disruption implements are described below.

B. Holding Blocks and Holding Block Loading Devices

In certain embodiments, cellular disruption components include holding block receiving areas that are structured to receive and position cellular disruption implement holding blocks. In some embodiments, holding blocks are structured to hold cellular disruption implements that can be placed in and removed from the holding blocks as desired. One advantage of these holding block embodiments is that the holding blocks can be re-used multiple times, e.g., using different cellular disruption implements each time. The invention also provides holding block loading devices that can be used to load cellular disruption implements into holding blocks in some of these embodiments. In other embodiments, holding blocks and cellular disruption implements are fabricated as integral units (i.e., the cellular disruption implements are not removable from the holding blocks). In some of these embodiments, the holding block with integral cellular disruption implements are intended to be disposable or consumable system components, whereas in others, these types of holding blocks can be re-used in multiple cellular disruption processes, e.g., after intervening sterilization or other processing steps have been performed on the holding blocks. In certain embodiments, holding blocks, whether with integral cellular disruption implements or not, are included in kits that can be, e.g., sold for use in the systems described herein.

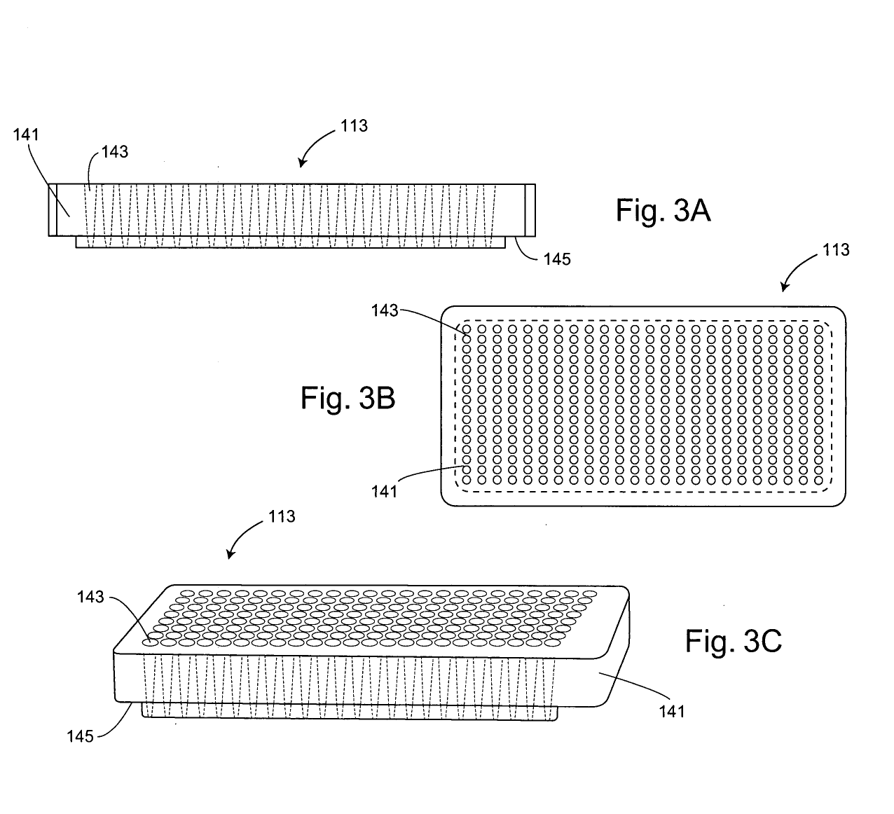

FIGS. 3A-C schematically depict one representative holding block embodiment. In particular, FIG. 3A schematically shows holding block 113 from a transparent side elevational view, while FIGS. 3B and C schematically depict holding block 113 from transparent top and transparent perspective views, respectively. As shown, holding block 113 includes body structure 141. There are multiple orifices 143 disposed through body structure 141. Orifices 143 are structured to receive and retain 384 cellular disruption implements 111 (see, e.g., FIG. 2A). Cellular disruption implements (e.g., radiation sources, electrical sources, thermal sources, mechanical disruption devices, etc.) are described further below. Holding blocks are optionally fabricated from many different types of materials (e.g., polymers, metals, metal alloys, etc.) using various fabrication techniques, such as injection molding and machining, among many others. Exemplary fabrication materials and techniques are described further.

Different orifice configurations, than the one depicted in FIGS. 3A-C, are also optionally utilized. In some embodiments, for example, holding blocks include orifice configurations that correspond to each well of other multi-well container formats (e.g., 12-well containers, 24-well containers, 48-well containers, 96-well containers, 192-well containers, 1536-well containers, etc.). In other embodiments, the orifice configuration of a holding block corresponds to only a subset of wells of a particular multi-well container, such as to every other row or column of wells, to every other well within a given row or column of wells, among many other possibilities that will be apparent to one of skill in the art to which this invention pertains. In certain embodiments, a holding block is structured to receive and retain only a single cellular disruption implement.

Cellular disruption implements, such as mechanical disruption devices, typically extend from holding block body structures sufficient distances or lengths (e.g., minimum lengths, etc.) so that the implements can contact the bottom surfaces of wells disposed in multi-well containers during operation of certain cellular disruption systems described herein. In these embodiments, the body structures typically substantially limit or prevent deflection of cellular disruption implements at regions other than those that extend from the body structures (e.g., in those regions disposed within orifices 143). In some embodiments, holding blocks not only accurately locate cellular disruption implements along the Z-axis, but also along the X- and Y-axes.

As further shown, for example, in FIGS. 3A and C, body structure 141 also includes retaining surface 145 that is received by bottom support 123 when holding block 113 is positioned in holding block receiving area 115 of automated cellular disruption system 100 (see, e.g., FIG. 2B).

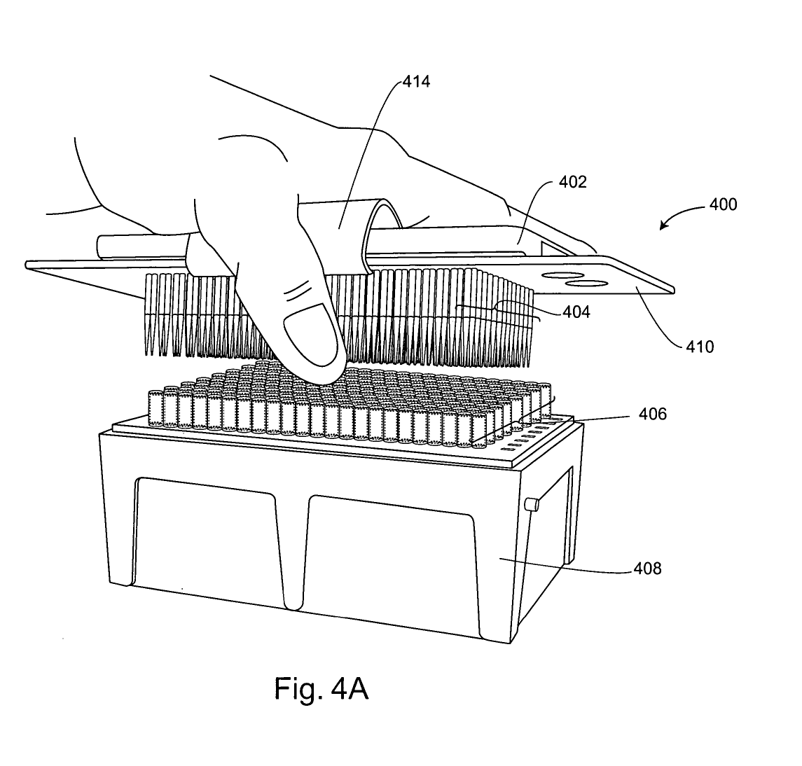

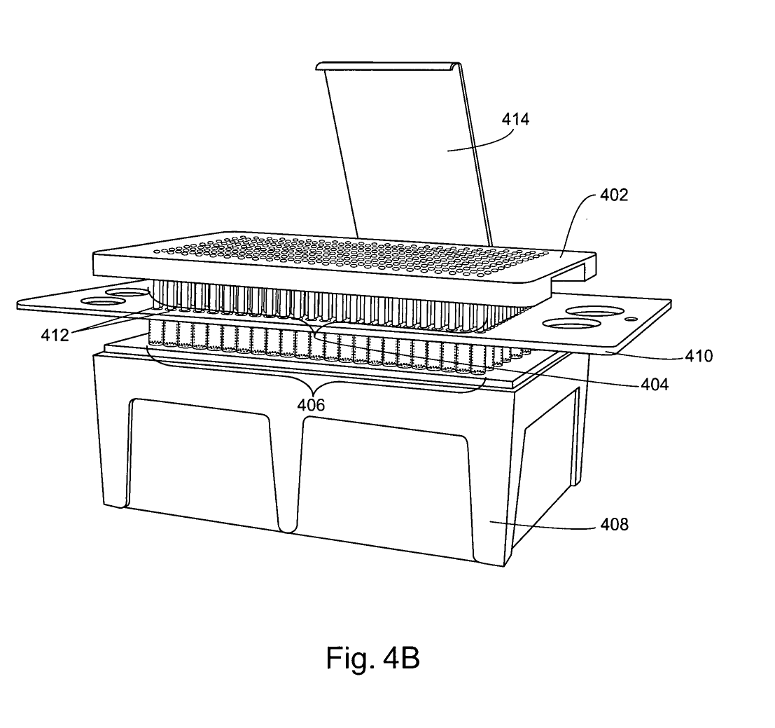

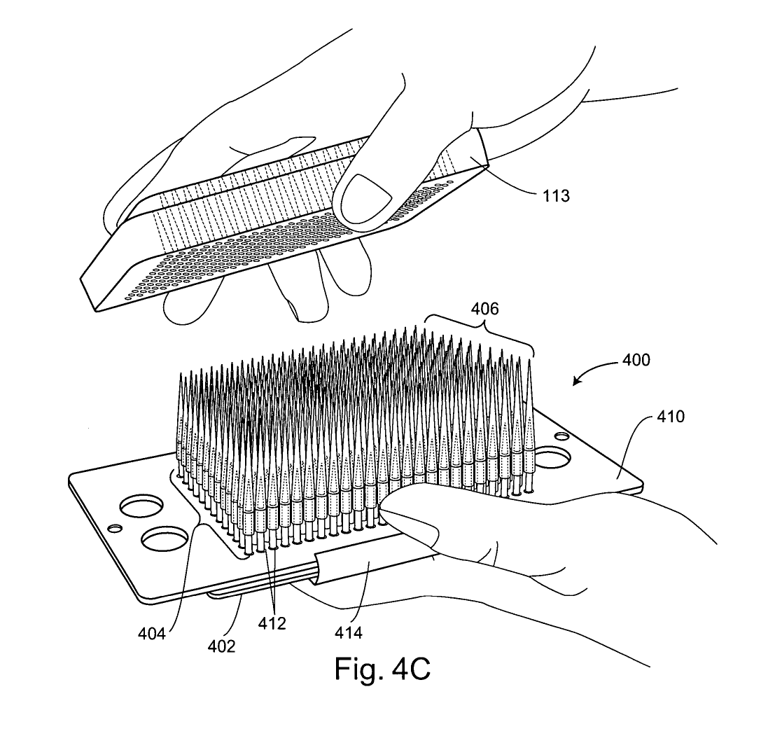

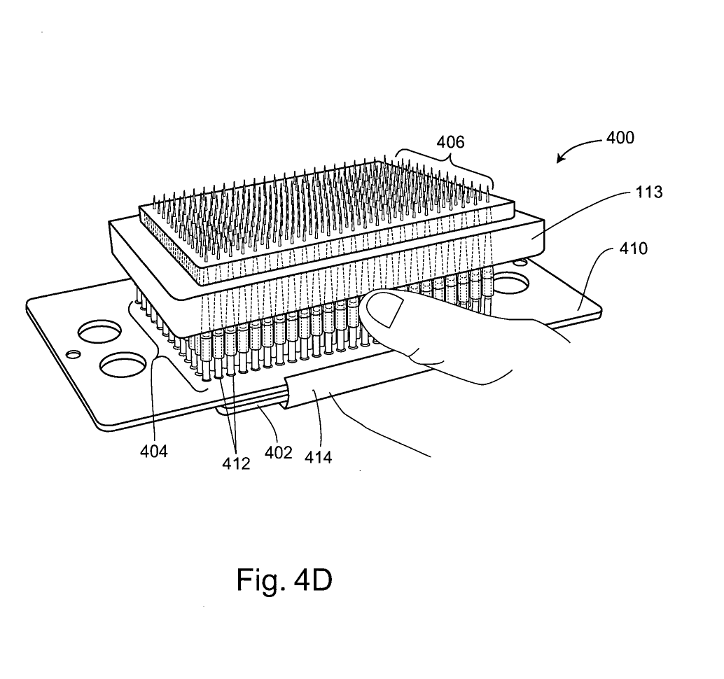

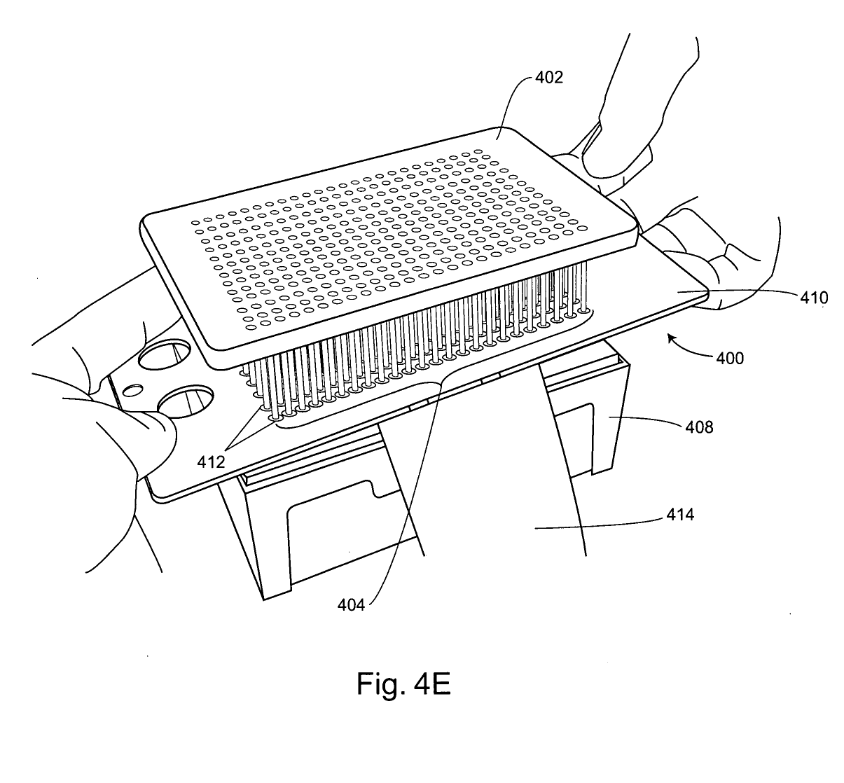

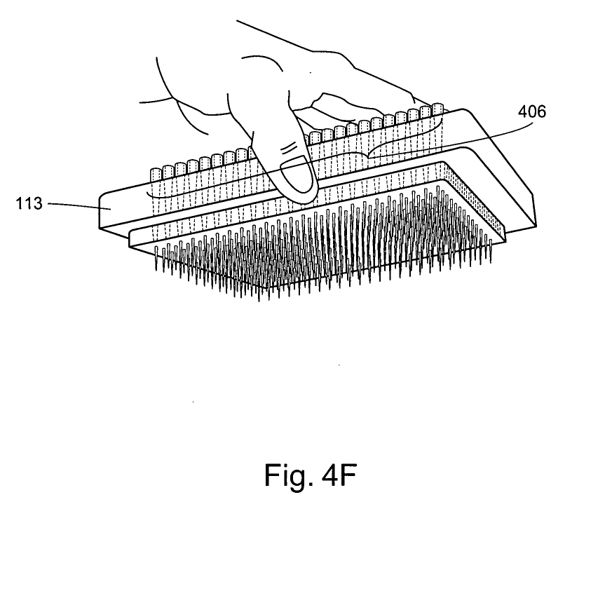

The invention also provides holding block loading devices that can be used to load cellular disruption implements into the orifices of holding blocks. FIGS. 4A-E schematically illustrate one holding block loading device embodiment. As shown, holding block loading device 400 includes support plate 402 and a plurality of protrusions 404 that protrude from a surface of support plate 402 and structured to engage pipette tips 406 disposed in pipette tip box 408, which hold pipette tips 406, e.g., prior to loading pipette tips 406 onto protrusions 404. In addition, protrusions 404 correspond to orifices 143 disposed through body structure 141 of holding block 113. The protrusions utilized in the holding block loading devices of the invention are generally non-fluid conveying pins or prongs having appropriate diameters to engage and retain the particular type of pipette tip used in a given application. As also shown, holding block loading device 400 also includes disengagement plate 410 having a plurality of holes 412 through which the plurality of protrusions 404 protrude. Disengagement plate 410 slides relative to protrusions 404, e.g., to disengage pipette tips 406 from protrusions 404 when desired. In some embodiments, holding block loading devices include, e.g., a resilient coupling (e.g., a spring, etc.) that couples support plates and disengagement plates to one another. As additionally shown, holding block loading device 400 also includes retaining mechanism 414 (shown as a latch) that is structured to retain disengagement plate 410 at a desired position relative to support plate 402, e.g., when protrusions 404 are being inserted into pipette tips 406 disposed in pipette tip box 408 by a user.

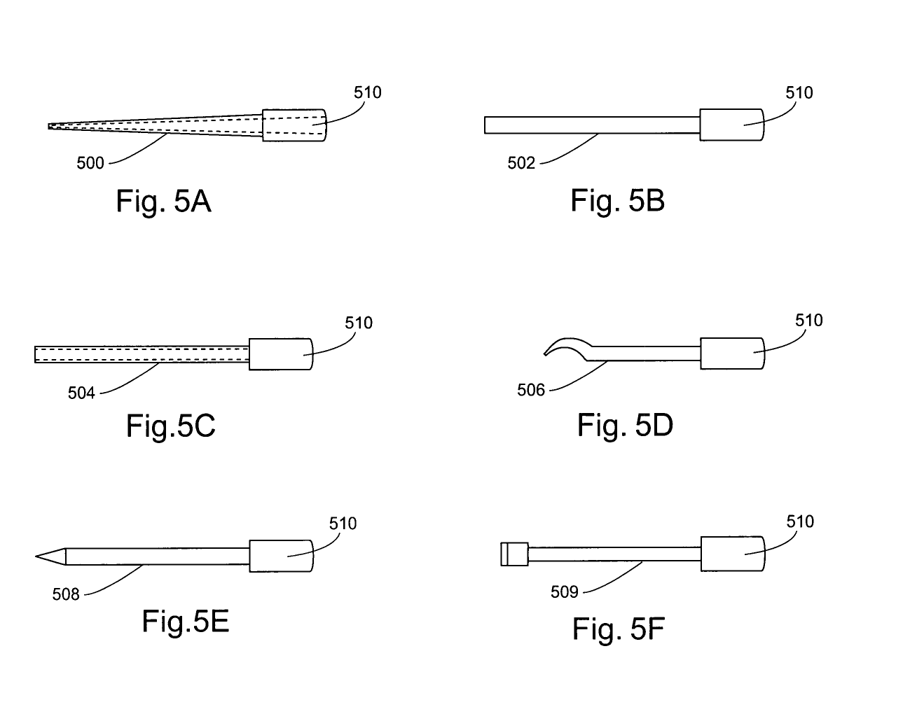

To further illustrate, FIGS. 4A-E also schematically depict an exemplary method of loading pipette tips 406 into holding block 113 prior to positioning holding block 113 in holding block receiving area 115 of automated cellular disruption system 100. More specifically, FIG. 4A schematically shows a user positioning holding block loading device 400 over pipette tip box 408 in preparation for engaging pipette tips 406. FIG. 4B schematically depicts protrusions 404 of holding block loading device 400 engaging pipette tips 406 in pipette tip box 408 after protrusions 404 have been inserted into pipette tips 406. FIG. 4C schematically illustrates the user positioning holding block 113 over pipette tips 406 disposed on holding block loading device 400 after pipette tips 406 have been removed from pipette tip box 408 in preparation for engaging pipette tips 406 in holding block 113. FIG. 4D schematically shows holding block 113 partially engaging pipette tips 406 loaded on protrusions 404 of holding block loading device 400. To more completely engage and locate pipette tips 406, the user typically pushes holding block 113 into contact with the collars of pipette tips 406 (see, e.g., collar 510 of pipette tip 500, which is schematically shown in FIG. 5A). As shown in FIG. 4E, the user typically disengages protrusions 404 of holding block loading device 400 from pipette tips 406 positioned in holding block 113 by inserting portions of pipette tips 406 back into pipette tip box 408 and pressing down on disengagement plate 410 to dislodge pipette tips 406 from protrusions 404. FIG. 4F schematically shows pipette tips 406 loaded in holding block 113 before holding block 113 is positioned in holding block receiving area 115 of automated cellular disruption system 100.

C. Cellular Disruption Implements

Many different types of cellular disruption implements are optionally utilized in the automated cellular disruption systems of the invention. Further, these implements can be configured (e.g., as in the holding blocks as described above) for use with essentially any type of container, including multi-well containers. Examples of the types of cellular disruption processes that can be performed using the systems of the invention, include mechanically disrupting cells, photobleaching cells, applying an electric field to cells, laser ablating cells, applying thermal energy to cells, exposing cells to ultra-violet radiation, among others known to those of skill in the art.

To illustrate, a variety of mechanical disruption devices are optionally used in these systems to disrupt cells by physically contacting the devices with the cells. For example, FIGS. 5A-E schematically show some of these devices from side elevational views. In particular, FIG. 5A schematically shows pipette tip 500, FIG. 5B schematically illustrates prong 502, FIG. 5C schematically depicts needle 504, FIG. 5D schematically depicts scraper 506, FIG. 5E schematically shows pin 508, and FIG. 5F schematically shows razor 509. In system embodiments that utilize holding blocks, cellular disruption implements typically include one or more locating features that are structured to locate the implements relative to the holding blocks. To illustrate, collars 510 schematically depicted in FIGS. 5A-E function as locating features. Mechanical disruption devices can typically be easily fabricated or are readily available in final or adaptable forms from various commercial suppliers known to persons of skill in the art. Fabrication techniques that are optionally utilized are described further below or otherwise known in the art. Examples of commercial suppliers of certain mechanical disruption devices, such as pipette tips, include Matrix Technologies Corp. (Hudson, N.H., USA), Millipore Corp. (Billerica, Mass., USA), Mettler-Toledo, Inc. (Columbus, Ohio, USA), and Greiner Bio-One, Inc. (Longwood, Fla., USA), among many others.

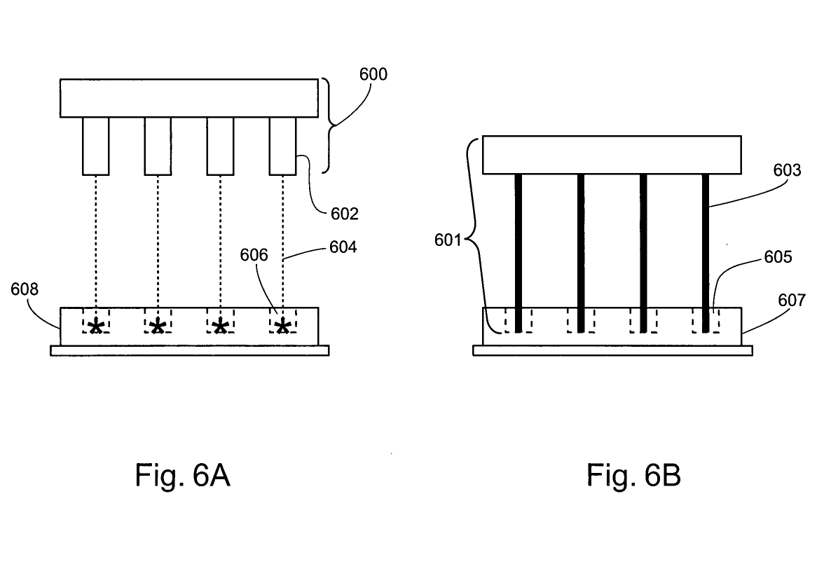

Other exemplary cellular disruption implements that are optionally used in the systems described herein include radiation sources, electrical sources, thermal sources, and the like. To illustrate, FIG. 6A schematically shows cellular disruption component 600, which comprises radiation sources 602 (shown as lasers) from a front elevational view. During operation, radiation 604 from radiation sources 602 disrupts cells disposed on the bottom surfaces of wells 606 of multi-well plates 608, e.g., as part of a laser ablation process. Another exemplary cellular disruption technique includes resistively heating materials within containers by flowing current through an electrode or other conductive component positioned within the container. As an example, FIG. 6B schematically shows cellular disruption component 601 that includes electrical or thermal source 603 (shown as electrodes) that flow current into fluids and/or cells disposed within wells 605 of multi-well plate 607 to resistively heat the fluid and/or cells disposed in wells 605, e.g., by dissipating energy through the electrical resistance of the electrodes, the fluid, and/or the cells, thereby effecting cellular disruption. In some of these embodiments, multiple electrodes (e.g., anodes and cathodes) are disposed in each well.

D. Translational Mechanisms

A variety of different translational mechanisms can be used in the systems of the invention to effect the movement cellular disruption components and/or container positioning components in one or more directions during cellular disruption processes, e.g., in substantially uniform modes. In some embodiments, for example, various types of devices including operably connected motors are utilized, such as linear actuators, air tables, X/Y-axis linear motion tables (e.g., operably connected to position feedback control drives, etc.), and the like. Linear actuators are generally devices that transform rotary motion into linear motion and typically include a motor connected to a ball or acme screw having a nut mounted in a telescopic tube. Optionally, air and hydraulic cylinders are utilized to effect the movement of system components. Typically, container positioning components or object holders are mounted on, e.g., single-axis or X/Y-axis linear motion tables. A representative single-axis linear motion component (see, translational mechanism 104) and a representative air table (see, air table 108) are schematically shown in FIG. 1, which is described further above.

Exemplary motors that are optionally utilized in the systems of the invention include, e.g., DC servomotors (e.g., brushless or gear motor types), AC servomotors (e.g., induction or gearmotor types), stepper motors, linear motors, or the like. Servomotors typically have an output shaft that can be positioned by sending a coded signal to the motor. As the input to the motor changes, the angular position of the output shaft changes as well. Stepper motors generally use a magnetic field to move a rotor. Stepping can typically be performed in full step, half step, or other fractional step increments. Voltage is applied to poles around the rotor. The voltage changes the polarity of each pole, and the resulting magnetic interaction between the poles and the rotor causes the rotor to move.

In some embodiments, the systems of the invention also include motor drives (e.g., AC motor drives, DC motor drives, servo drives, stepper drives, etc.), which act as interfaces between controllers and motors. In certain embodiments, motor drives include integrated motion control features. For example, servo drives typically provide electrical drive output to servo motors in closed-loop motion control systems, where position feedback and corrective signals optimize position and speed accuracy. Servo drives with integrated motion control circuitry and/or software that accept feedback, provide compensation and corrective signals, and optimizes position, velocity, and acceleration.

Suitable linear actuators, linear motion tables, motors and/or motor drives are generally available from many different commercial suppliers including, e.g., linear actuators (SKF Group, Göteborg, Sweden), IAI America, Inc. (Torrance, Calif., USA), MPC Products Corporation (Skokie, Ill., USA), Yaskawa Electric America, Inc. (Waukegan, Ill., USA), AMK Drives & Controls, Inc. (Richmond, Va., USA), Enprotech Automation Services (Ann Arbor, Mich., USA), Aerotech, Inc. (Pittsburgh, Pa., USA), Quicksilver Controls, Inc. (Covina, Calif., USA), NC Servo Technology Corp. (Westland, Mich., USA), HD Systems Inc. (Hauppauge, N.Y., USA), ISL Products International, Ltd. (Syosset, N.Y., USA), and the like. X/Y-axis linear motion tables, motors, and motor drives are also described in, e.g., U.S. Pat. Appl. Pub; No. 20050163637, entitled “MATERIAL CONVEYING SYSTEMS, COMPUTER PROGRAM PRODUCTS, AND METHODS” filed Dec. 1, 2004 by Chang et al., Polka, Motors and Drives, ISA (2002) and Hendershot et al., Design of Brushless Permanent-Magnet Motors, Magna Physics Publishing (1994), which are each incorporated by reference.

E. Container Positioning Components

The automated cellular disruption systems of the invention typically include container positioning components that are structured to position containers relative to cellular disruption components. In some embodiments, these positioning components are mounted on translational mechanisms, such as air tables, X/Y-axis linear motion tables, or the like, whereas in other embodiments, container positioning components are mounted or otherwise placed in fixed positions relative to the cellular disruption components. In certain components, a container positioning component simply comprises a support surface (e.g., the top surface of a table, a bench, or the like) on or above which one or more other components (e.g., cellular disruption components, linear actuators or linear motion tables coupled with cellular disruption components, controllers, etc.) of the system are positioned.

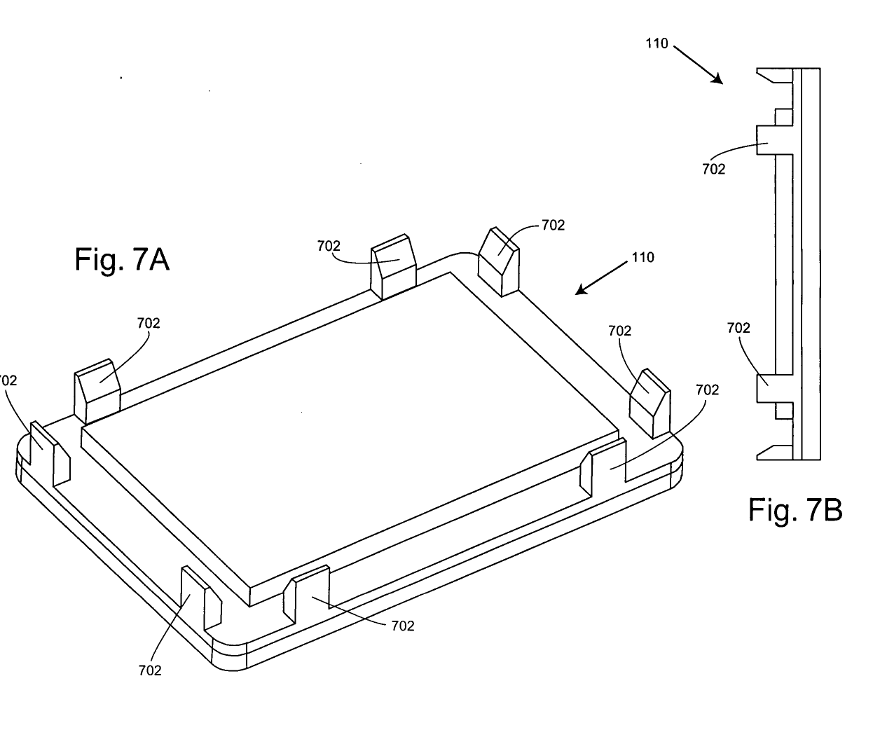

An example of a container positioning component is schematically depicted in FIGS. 7A and B from perspective and side elevational views, respectively. As shown, container nest 110 includes alignment features 702 formed on a top surface (e.g., via machining, molding, etc.). Alignment features 702 are used to align containers when they are placed into container nest 110. Although container nest 110 is shown attached to a translational mechanism in FIG. 1, it can also be placed at a fixed position relative to a cellular disruption component in other embodiments, such as those in which the cellular disruption component is attached to a translational components that is configured to move along multiple translational axes. Although other materials are optionally utilized, container nest 110 is fabricated from stainless steel in certain embodiments.

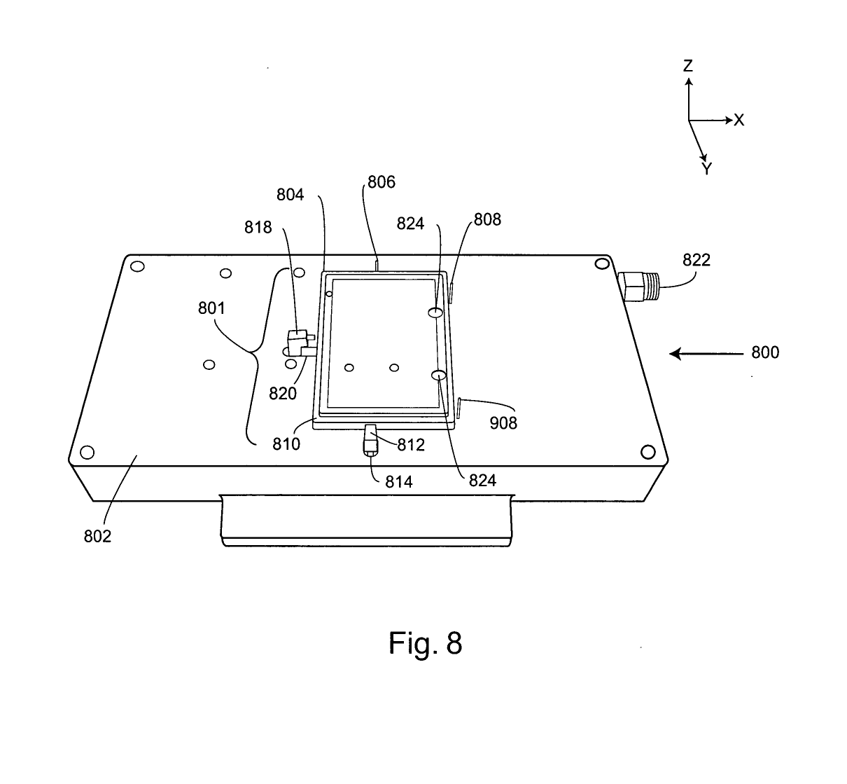

For positioning along two different axes, the container positioning components of the systems of the invention generally have one or more alignment members positioned to receive and align, e.g., each of the two axes of a multi-well container. For example, FIG. 8 shows a top perspective view of container positioning component 800 that can be used in the automated cellular disruption systems described herein. Container positioning component 800 is optionally placed at a fixed position or attached to a translational mechanism. As shown in FIG. 8, container station 801 is disposed on support structure 802 of container positioning component 800. Support structure 802 supports vacuum plate 804. Protrusions 806 and 808 function as alignment members. The illustrated embodiment of container station 801 has two x-axis protrusions 808 and one y-axis protrusion 806 extending from support structure 802. Accordingly, x-axis protrusions 808 and y-axis protrusion 806 are fixedly positioned relative to the vacuum plate 804, which, in this embodiment, acts to hold a multi-well container in position once it has been positioned. X-axis locating protrusions 808 are constructed to cooperate with an x-axis surface of a multi-well container (e.g., a x-axis wall of a microtiter plate), while y-axis protrusion 806 is constructed to cooperate with an y-axis surface of the container (e.g., a y-axis wall of a microtiter plate).

The alignment members can be, for example, locating pins, tabs, ridges, recesses, or a wall surface, and the like. In some embodiments, an alignment member includes a curved surface that contacts a properly positioned multi-well container. The use of a curved surface minimizes the effect of, for example, roughness of the container surface that contacts the alignment member. The use of two alignment members along one axis and one alignment member along the second axis, as shown in FIG. 8, is another approach to minimize the effect of surface irregularities on the proper positioning of the container. The multi-well container contacts three points along the surface of the container, so proper alignment is not dependent upon the entire container surface being regular.

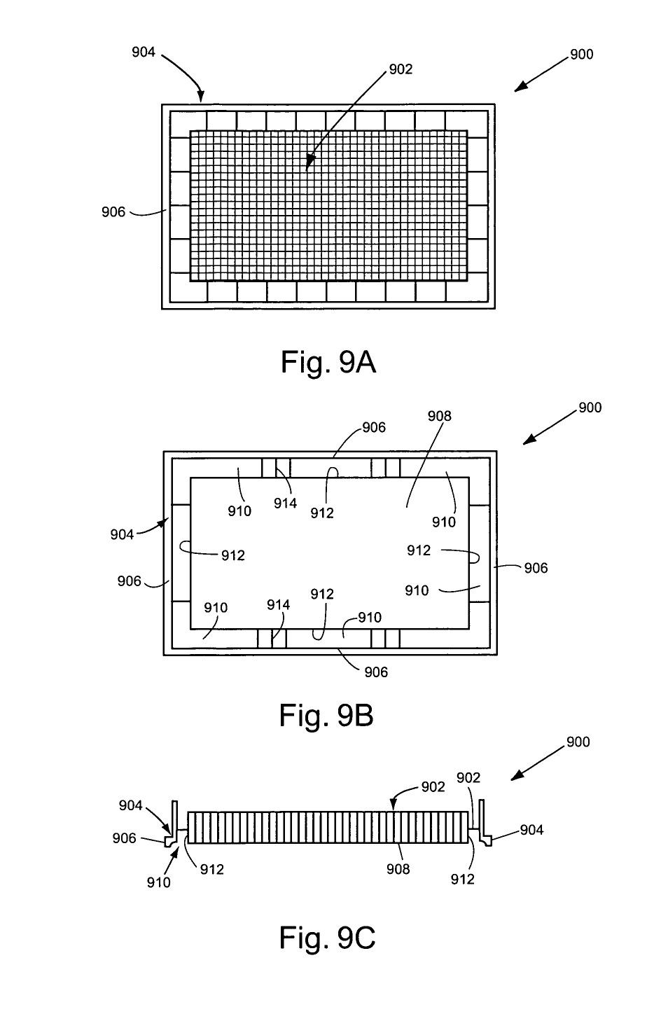

Certain aspects of the invention apply specifically to the positioning of microtiter plates, e.g., when used in a cellular motility assay or the like. To illustrate, microtiter plate 900 is shown in FIGS. 9A-C. As shown, microtiter plate 900 comprises well area 902, which has many individual sample wells for holding samples and reagents. Microtiter plates are available in a wide variety of sample well configurations, including commonly available plates with 6, 12, 24, 48, 96, 192, 384, 768, 1536, 9600, or more wells. It will be appreciated that microtiter plates are available from a various manufacturers including, e.g., Greiner America Corp. (Lake Mary, Fla., USA), Nalge Nunc International (Rochester, N.Y., USA), and the like. Microtiter plate 900 has outer wall 904 having registration edge 906 at its bottom. In addition, microtiter plate 900 includes bottom surface 908 below the well area on the plate's bottom side. Bottom surface 908 is separated from outer wall 904 by alignment member receiving area 910. Alignment member receiving area 910 is bounded by a surface of outer wall 904 and by inner wall 912 at the edge of bottom surface 908. Although there may be some lateral supports 914 in alignment member receiving area 910, these areas are generally open between inner wall 912 and an inner surface of the outer wall 904.

In certain embodiments, to position a microtiter plate the alignment members of the container station are optionally arranged to cooperate with inner wall 912 of the microtiter plate. Inner wall 912 is advantageously used, as inner wall 912 is typically more accurately formed and is more closely associated with the perimeter of the sample well area, as compared to an outer wall of plate 900, such as wall 904. Accordingly, aligning an inner wall (e.g., inner wall 912) of a microtiter plate relative to alignment members is generally preferred to aligning with an outer wall, such as wall 904. The increased positioning precision that is obtained by using an inner wall as the alignment surface makes possible the use of high-density microtiter plates, such as 384-well plates, 1536-well plates, etc. Further, by having the alignment members (e.g., alignment protrusions 806 and 808) cooperate with an inner wall 912 of plate 900, minimal structures are needed adjacent the outside of the plate. In such a manner, a robotic arm or other transport device is able to readily access plate 900. Having the protrusions positioned adjacent inner wall 912 thereby facilitates translocating plate 900. However, it will be appreciated that the alignment members or protrusions can be placed in alternative positions and still facilitate the precise positioning of the plate.

In some embodiments, container positioning components include one or more movable members. The movable members function to move a container against one or more alignment members. For example, once a multi-well container is placed in the general location of the alignment members, the movable members (termed “pushers” herein) move the container so that an alignment surface of the container is in contact with one or more of the alignment members of the positioning component. The positioning component can have pushers for positioning of the container along one or more axes. For example, a positioning component will often have one or more pushers that position a container along an x-axis, and one or more additional pushers that position the container along a y-axis. The pushers can be moved by means known to those of skill in the art. For example, air cylinders, springs, pistons, elastic members, electromagnets or other magnets, gear drives, and the like, or combinations thereof, are suitable for moving the pushers so as to move containers into a desired position.

One embodiment of a container station of a container positioning component having pushers for positioning a microtiter plate along both the x-axis and the y-axis is shown in FIG. 8. When the microtiter plate is generally positioned adjacent the x- and y-axis protrusions, the bottom surface of the microtiter plate is directly above top surface 810 of vacuum plate 804. Y-axis pusher 812, which extends through slot 814 in support structure 802, is used to apply pressure to a y-axis side wall of the microtiter plate. Sufficient force is applied to the plate to push the microtiter plate against y-axis protrusion 806. When the microtiter plate is pushed against y-axis protrusion 806, x-axis pusher 818, which extends through slot 820 of support structure 802, is used to push an x-axis wall of the microtiter plate towards x-axis protrusions 808. In this manner, the microtiter plate is accurately and precisely positioned relative both the x-axis and y-axis protrusions. It is sometimes advantageous, although not necessary, to have one or more of the pushers contact an inner wall of a microtiter plate rather than an outer wall. With this arrangement, the alignment members and pushers are underneath the microtiter plate. This leaves the area surrounding the exterior of the plate free of protrusions that could otherwise interfere with other devices that, for example, place the microtiter plate on the support.

As referred to above, the container positioning component embodiment shown in FIG. 8 includes vacuum plate 804 that functions as a retaining device to hold a properly positioned container in a desired position. With both y-axis pusher 812 and x-axis pusher 818 applying sufficient force to precisely place the microtiter plate, a vacuum source (not shown) applies a vacuum through vacuum line 822 into vacuum openings or holes 824. Air source (not shown) applies air pressure through an air line (not shown) to effect movement of the pushers.

As referred to above, container positioning components are optionally attached to X/Y-axis linear motion tables operably connected to position feedback control drives that control movement of the X/Y-axis linear motion tables along X- and Y-axes. In certain embodiments, linear motion tables are configured to move only along a single axis, such as an X-axis or a Y-axis.

Various other container positioning components or portions thereof can be utilized or adapted for use in the systems of the invention. Some of these container positioning components are also described in, e.g., International Publication No. WO 01/96880, entitled “AUTOMATED PRECISION OBJECT HOLDER,” filed Jun. 15, 2001 by Mainquist et al., U.S. patent application Ser. No. 10/911,238, entitled “MULTI-WELL CONTAINER POSITIONING DEVICES AND RELATED SYSTEMS AND METHODS,” filed Aug. 3, 2004 by Evans, U.S. patent application Ser. No. 10/911,388, entitled “NON-PRESSURE BASED FLUID TRANSFER IN ASSAY DETECTION SYSTEMS AND RELATED METHODS,” filed Aug. 3, 2004 by Evans et al., and U.S. Provisional Patent Application No. 60/645,502, entitled “TI-WELL CONTAINER POSITIONING DEVICES, SYSTEMS, COMPUTER PROGRAM PRODUCTS, AND METHODS,” filed Jan. 19, 2005 by Chang et al., which are each incorporated by reference.

F. Controllers

The automated cellular disruption systems of the invention also typically include controllers that are operably connected to, e.g., cellular disruption components, translational mechanisms, container positioning components, etc. and/or to other additional system components when they are included (e.g., robotic gripping components, assaying components, cell culture components, material handling components, removal components, dispensing components, incubation components, container storage components, detection components, etc.) to control the operation of those components. More specifically, controllers are generally included either as separate or integral system components that are utilized, e.g., to open and close certain cellular disruption components, to move cellular disruption components and/or container positioning components relative to one another in substantially uniform modes, to move robotic gripping devices, etc. Controllers and/or other system components is/are optionally coupled to an appropriately programmed processor, computer, digital device, or other information appliance (e.g., including an analog to digital or digital to analog converter as needed), which functions to instruct the operation of these instruments in accordance with preprogrammed or user input instructions, receive data and information from these instruments, and interpret, manipulate and report this information to the user. One controller embodiment is schematically depicted in FIG. 1 (see, controller 112).

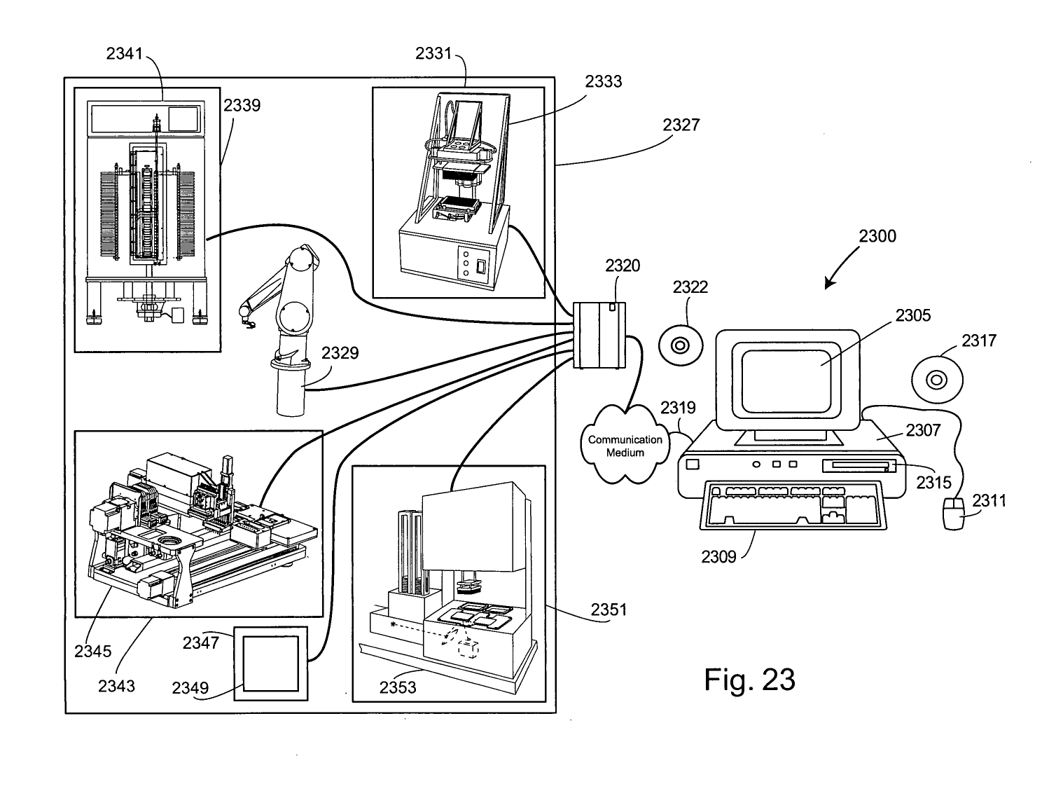

Any controller or computer optionally includes a monitor that is often a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user. An exemplary computer is schematically shown in FIG. 23, which is described further below.

The computer typically includes appropriate software for receiving user instructions, either in the form of user input into a set of parameter fields, e.g., in a GUI, or in the form of preprogrammed instructions, e.g., preprogrammed for a variety of different specific operations. The software then converts these instructions to appropriate language for instructing the operation of one or more controllers to carry out the desired operation, e.g., varying or selecting the rate or mode of movement of various system components, directing translation of robotic gripping devices, fluid dispensing heads, or of one or more multi-well containers or other vessels, or the like. The computer then receives the data from, e.g., sensors/detectors included within the system, and interprets the data, either provides it in a user understood format, or uses that data to initiate further controller instructions, in accordance with the programming, e.g., such as in monitoring incubation temperatures, detectable signal intensity, or the like.

To further illustrate, the automated cellular disruption systems of the invention generally include system software that effects the control of cellular disruption component and/or container positioning component movement in substantially uniform modes. For example, the software typically includes logic instructions for receiving user input in the form of substantially uniform mode parameter selections. Types of selectable parameters that are generally included are the container format being utilized (e.g., number of wells in a multi-well container, standard or non-standard multi-well container, etc.), and distances, pathways, and rates of component movement relative to one another. In some embodiments, for example, the user inputs a multi-well container format and the software directs the cellular disruption component and/or container positioning to move a set distance that is a fraction of a cross-sectional dimension of a well of the container according to the input multi-well container format. In other embodiments, the user selects these distances directly. In certain embodiments, systems are preprogrammed with selectable pathways of cellular disruption component and/or container positioning movement to effect a given pattern of cellular disruption (e.g., a pattern physical cellular disruption using a mechanical disruption device, a laser ablation pattern, etc.) within a given container, such as a rectilinear or curvilinear pattern. When mechanical disruption devices are used, the software optionally includes instructions that effect a level of force (user selectable or preprogrammed) applied by cellular disruption components and/or container positioning components on containers, e.g., such that the mechanical disruption devices deflect upon contacting surfaces of containers. In some embodiments, for example, systems are configured to apply a unit load sufficient to push the bottom walls of multi-well container wells between about 0.20 mm and about 0.55 mm downward relative to initial positions of those walls when the mechanical disruption devices contact the walls. Computer program products that can be used in the systems of the invention are also described below.

The computer can be, e.g., a PC (Intel x86 or Pentium chip-compatible DOS™, OS2™, WINDOWS™, WINDOWS NT™, WINDOWS95™, WINDOWS98™, WINDOWS2000™, WINDOWS XP™, LINUX-based machine, a MACINTOSH™, Power PC, or a UNIX-based (e.g., SUN™ work station) machine) or other common commercially available computer that is known to one of skill in the art. Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Paradox™) can be adapted to the present invention. Software for performing, e.g., component movement, multi-well container positioning, fluid removal from selected wells of a multi-well container, etc. is optionally constructed by one of skill in the art using a standard programming language such as AppleScript, C, C+, Perl, Visual basic, Fortran, Basic, Java, or the like.

In certain embodiments, the bar codes described above or other labels affixed to the containers are optionally used to provide a container or sample inventory, e.g., that is tracked by a controller for the systems of the invention. The inventory typically keeps track of what samples and/or containers are in the system, as well as their location and status within the system. In addition, information can be transferred to a central controller, e.g., a PC, that coordinates locations with resulting data from various processes to provide an inventory combined with assay results. Typically, the systems include container location databases operably connected to controllers. These databases generally include entries that correspond to locations of containers in the system or other desired information.

G. Computer Program Products

It will be appreciated that various embodiments of the present invention provide methods and/or systems for disrupting cell populations disposed in containers that can be implemented at least in part on a general purpose or special purpose information handling appliance using a suitable programming language such as Java, C++, C#, Perl, Python, Cobol, C, Pascal, Fortran, PL1, LISP, assembly, etc., and any suitable data or formatting specifications, such as HTML, XML, dHTML, tab-delimited text, binary, etc. In the interest of clarity, not all features of an actual implementation are described herein. It will be understood that in the development of any such actual implementation (as in any software development project), numerous implementation-specific decisions must be made to achieve the developers' specific goals and subgoals, such as compliance with system-related and/or business-related constraints, which will vary from one implementation to another. Moreover, it will be appreciated that such a development effort might be complex and time-consuming, but would nevertheless be a routine undertaking of software engineering for those of ordinary skill having the benefit of this disclosure.

To generally illustrate certain control software that can implement aspects of the invention, one computer program product includes a computer readable medium having logic instructions for moving a cellular disruption component and/or a container positioning component of an automated cellular disruption system relative to one another such that the cellular disruption component disrupts cells disposed in a container positioned by the container positioning component. In certain embodiments, the computer readable medium comprises at least one logic instruction for receiving at least one input parameter selected from, e.g., a distance of cellular disruption component and/or container positioning component movement, a pathway of cellular disruption component and/or container positioning component movement, a rate of cellular disruption component and/or container positioning component movement, a level of force applied by the cellular disruption component and/or the container positioning component on the container, a container format, and the like. In some embodiments, the computer readable medium comprises at least one logic instruction for moving the cellular disruption component and/or the container positioning component along a first axis such that at least one cellular disruption implement of the cellular disruption component contacts at least one surface of the container comprising the cells when the container is positioned relative to the automated cellular disruption system, and moving the cellular disruption component and/or the container positioning component along at least a second axis to disrupt the cells when the container is positioned by the container positioning component and the cellular disruption implement contacts the surface of the container. In these embodiments, the computer readable medium optionally includes at least one logic instruction for contacting the cellular disruption implement with the surface of the container with sufficient force to deflect the cellular disruption implement away from the first axis. Exemplary computer readable media include, e.g., a CD-ROM, a floppy disk, a tape, a flash memory device or component, a system memory device or component, a hard drive, a data signal embodied in a carrier wave, and the like.

H. Additional System Components

The automated cellular disruption systems described herein optionally include one or more additional components, which together form expanded automated systems that can be used in a wide range of applications, including high-throughput cell-based compound profiling applications. These systems are typically highly automated with minimal user intervention for repeated usage at high throughput in, e.g., laboratory and industrial settings. To illustrate, certain other automated tissue culturing or compound profiling components or sub-systems are included to automate the process of cell seeding, incubation, trypsination, cell counting and viability determination, splitting of cell lines, collection and plating of cells, and the like. Examples of these additional components include assaying components, detection components, robotic gripping components, material handling components, incubation components, refrigeration components, container storage components, etc. Some of these additional components are described further below. Many of these as well as other additional components that are optionally included in the systems of the invention are also described in, e.g., U.S. Provisional Patent Application No. 60/664,640, entitled “COMPOUND PROFILING DEVICES, SYSTEMS, AND RELATED METHODS”, filed Mar. 22, 2005 by Chang et al., and U.S. Provisional Patent Application No. 60/680,132, entitled “COMPOUND PROFILING DEVICES, SYSTEMS, AND RELATED METHODS”, filed May 11, 2005 by Chang et al., which are both incorporated by reference.

1. Assaying Components

The systems of the invention optionally include assaying components that can support a broad range of assay formats, including screens for compounds with desired properties. In some embodiments, for example, the assaying components include non-pressure-based fluid transfer probes, such as pin tools. These assaying components are optionally used to transfer test compounds or other test reagents from test reagent plates into assay plates (e.g., assay plates that include 96-wells, 384-wells, 1536-wells, or even higher well densities). Depending on the particular assay being performed, cells are typically added to the assay plates either before or after test compounds are transferred to these plates. Assaying components that are optionally adapted for use in the systems of the present invention are also described in, e.g., U.S. patent application Ser. No. 10/911,388, entitled “NON—PRESSURE BASED FLUID TRANSFER IN ASSAY DETECTION SYSTEMS AND RELATED METHODS,” filed Aug. 3, 2004 by Evans et al., which is incorporated by reference.

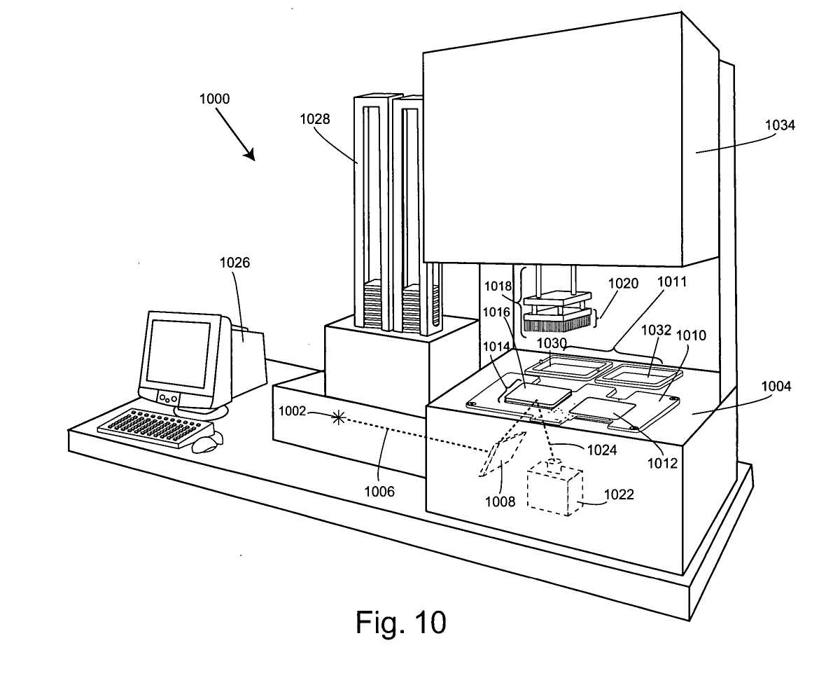

To further illustrate, FIG. 10 schematically shows an assaying component from a perspective view according to one embodiment of the invention. As shown, assaying component 1000 includes electromagnetic radiation source 1002, which is schematically depicted as a laser. Other electromagnetic radiation sources are also optionally adapted for use in the systems of the invention, including electroluminescence devices, laser diodes, light-emitting diodes (LEDs), incandescent lamps, arc lamps, flash lamps, fluorescent lamps, and the like. Assaying component 1000 also includes sample assaying region 1004, which is configured to receive source electromagnetic radiation 1006 from electromagnetic radiation source 1002 via mirror 1008. Various optical systems are optionally utilized or adapted for use in the systems of the invention. Exemplary optical systems are described or referred to herein. Other suitable optical systems are known in the art and will be apparent to those of skill in the art.

In some embodiments, sample assaying region 1004 includes container positioning component or device 1010, which includes container stations 1012 and 1014 that are each structured to position container 1016 (shown as a multi-well container) relative to fluid transfer device 1018. Fluid transfer device 1018 includes non-pressure-based fluid transfer probe 1020 (shown as a pin tool). Sample assaying region 1004 also includes transfer probe washing station 1011, which includes wash reservoirs 1030 and 1032 for washing non-pressure-based fluid transfer probe 1020. Fluid transfer device 1018 is configured to transfer fluid in at least one selected region (e.g., sample assaying region 1004, as shown) of assaying component 1000. In certain embodiments, non-pressure-based fluid transfer probe 1020 is removably attached to a chassis of fluid transfer device 1018. As also shown, assaying component 1000 also includes detector 1022 configured to detect sample electromagnetic radiation 1024 received from sample assaying region 1004. Various detectors are optionally adapted for use in the assaying components of the invention including, e.g., charge-coupled devices (CCDs), intensified CCDs, photomultiplier tubes (PMTs), photodiodes, avalanche photodiodes, etc. Hood 1034 of assaying component 1000 moves to enclose sample assaying region 1004 to exclude, e.g., electromagnetic radiation other than source and sample electromagnetic radiation 1006 and 1024, respectively, or other contaminates that may bias assay results from sample assaying region 1004. In certain embodiments, fluid transfer devices and detectors are included in separate stations of the systems of the invention.

Assaying component 1000 also includes controller 1026 (shown as computer) that is typically operably connected to, e.g., electromagnetic radiation source 1002, fluid transfer device 1018, and detector 1022. Optionally, controller 1026 is also operably connected to other system components. The controllers of the invention typically include at least one logic device (e.g., a computer such as the one illustrated in FIG. 10) having one or more logic instructions that direct operation of one or more components of the system. Also shown is container storage component 1028, which stores containers before and/or after being assayed.

2. Detection Components

The systems of the invention also generally include detectors or detection components that are structured to detect detectable signals produced, e.g., in the wells of multi-well containers, in cell culture flasks, in samples aliquots taken from cell culture flasks, or the like. As described above, for example, detectors are typically included in the assaying components of the systems of the invention. Optionally, other detection components are included in these systems in addition to or in lieu of the assaying components described above.

To illustrate, suitable signal detectors that are optionally utilized in the systems of the invention detect, e.g., fluorescence, phosphorescence, radioactivity, mass, concentration (e.g., reagent concentrations, cellular concentrations or cell counts, etc.), pH, charge, absorbance, refractive index, luminescence, temperature, magnetism, or the like. In one exemplary embodiment, an ACQUEST™ workstation (Molecular Devices Corp., Sunnyvale, Calif., USA) is included as a system component. These workstations typically include multi-mode readers and modified nests for robotic access. In some embodiments, the systems of the invention also include FACS arrays or other cell counting components. Examples of these components that are optionally adapted for use in the systems described herein include the BD FACSArray™ bioanalyzer system (BD Biosciences, San Jose, Calif., USA), the MetaMorph® Imaging System (Universal Imaging Corporation™ a subsidiary of Molecular Devices, Downingtown, Pa., USA), or the like. In certain embodiments, cells are photographed in multi-well containers using fluorescent microscopes. Certain fluorescent microscopes that are optionally used or adapted for use in the systems of the invention are available from, e.g., Quantitative 3-Dimensional Microscopy (Q3DM), Inc. (San Diego, Calif., USA).

Detectors optionally monitor one or a plurality of signals from upstream and/or downstream of the performance of, e.g., a given assay or processing step. For example, the detector optionally monitors a plurality of optical signals, which correspond in position to “real time” results. Example detectors or sensors include photomultiplier tubes, CCD arrays, optical sensors, temperature sensors, pressure sensors, pH sensors, conductivity sensors, scanning detectors, or the like. Each of these as well as other types of sensors is optionally readily incorporated into the systems described herein. Detectors are optionally configured to move relative to multi-well containers, cell culture flasks, or other components, or alternatively, multi-well containers, cell culture flasks, or other components are configured to move relative to the detector. In certain embodiments, for example, detection components are coupled to translation components that move the detection components relative to multi-well containers, cell culture flasks, or other containers positioned on object holders or container positioning devices described herein. Optionally, the systems of the present invention include multiple detectors. In these systems, such detectors are typically placed either in or adjacent to, e.g., a multi-well container or other vessel, such that the detector is within sensory communication with the multi-well container or other vessel (i.e., the detector is capable of detecting the property of the plate or vessel or portion thereof, the contents of a portion of the plate or vessel, or the like, for which that detector is intended).

Detectors optionally include or are operably linked to a computer, e.g., which has system software for converting detector signal information into assay result information or the like. For example, detectors optionally exist as separate units, or are integrated with controllers into a single instrument. Integration of these functions into a single unit facilitates connection of these instruments with the computer, by permitting the use of few or a single communication port(s) for transmitting information between system components. Computers and controllers are described further above. Detection components that are optionally included in the systems of the invention are described further in, e.g., Skoog et al., Principles of Instrumental Analysis, 5^{th} Ed., Harcourt Brace College Publishers (1998) and Currell, Analytical Instrumentation: Performance Characteristics and Quality, John Wiley & Sons, Inc. (2000), which are both incorporated by reference.

3. Robotic Gripping Components

The systems of the invention typically include one or more robotic gripping components that, at least in part, effect system automation. Typically, these components are configured for rotation about an axis with a rotational range of about 360 degrees. In addition, these robotic components generally adjust vertically and horizontally to align with relatively higher or lower work positions. Moreover, these rotational robotic components typically have a robotic arm that extend and retract from the robot's rotational axis. Accordingly, each rotational robot has an associated rotational reach, e.g., defining how far out from the rotational axis the robot is capable of operating. This rotational reach defines a work perimeter, e.g., a circular work perimeter, for that robot. Other system components, such as a cellular disruption system of the invention, are typically positioned within the work perimeter of a given robotic gripping component so that robotic component can transfer containers or other items between different system components. Work perimeters and related system configurations that are optionally adapted for use with the systems of the present invention are also described in, e.g., U.S. Patent Publication No. 2002/0090320, entitled “HIGH THROUGHPUT PROCESSING SYSTEM AND METHOD OF USING,” filed Oct. 15, 2001 by Burow et al., which is incorporated by reference.

In addition, a robotic arm typically includes a robotic gripper mechanism. For example, a gripper mechanism is used to grasp objects for transport between selected positions with a system. In certain embodiments, for example, gripper mechanisms are configured to grasp multi-well containers. Gripper mechanisms are also optionally configured to grasp other types of objects, including without limitation, custom sample holders, reaction vessels, reaction blocks, cell culture containers or flasks, crucibles, petri dishes, test tubes, test tube arrays, and vial arrays, among many others. Robotic arms and gripper mechanisms are typically operated pneumatically, hydraulically, magnetically, or by other means known to persons of skill in the art. Optionally, gripper mechanisms are coupled to robotic arms via a breakaway or other deflectable member that is structured to deflect when the gripper mechanism contacts an object with a force greater than a preset force, e.g., to minimize the risk of damage to the rotational robot and the object. Exemplary robotic gripping devices that are optionally adapted for use in the systems of the invention are described further in, e.g., U.S. Pat. No. 6,592,324, entitled “GRIPPER MECHANISM,” issued Jul. 15, 2003 to Downs et al. and International Publication No. WO 02/068157, entitled “GRIPPING MECHANISMS, APPARATUS, AND METHODS,” filed Feb. 26, 2002 by Downs et al., which are both incorporated by reference.

In some embodiments, the robotic gripping devices include sensors (e.g., optical sensors, etc.), e.g., for detecting containers or other objects being transported and the direction a particular sample container should be inserted into or onto a device, such as a container positioning component, a plate reader, etc. In addition, a sensor optionally determines a location of gripper mechanisms relative to objects to be transported.

Suitable robots are available from various commercial suppliers known in the art. In some embodiments, for example, Stäubli RX-60 robots (provided by Stäubli Corporation of South Carolina, U.S.A.) are utilized in the systems of the invention. Such robots are highly accurate and precise, e.g., typically to within about one one-thousandth of an inch. Other robot models from this or other suppliers are also optionally used. A variety of other robotic instrumentation that is optionally adapted for use with the present invention is available from, e.g., the Zymark Corporation (Hopkinton, Mass., USA), which utilize various Zymate systems, which can include, e.g., robotics and fluid handling modules. Similarly, the common ORCA® robot, which is used in a variety of laboratory systems, e.g., for microtiter tray manipulation, is also commercially available, e.g., from Beckman Coulter, Inc. (Fullerton, Calif., USA).

The robots and associated work perimeters and other system component station locations are typically attached to one or more frames that support the system components. To illustrate, weldments, aluminum extrusions, etc. are optionally used to provide support frames with optics table tops or other support surfaces for mounting various devices or systems, e.g., cellular disruption systems, cell culture passaging stations, incubators, detectors, and the like. Table tops such are these are commercially available from various suppliers, including Melles Griot, Inc. (Carlsbad, Calif., USA).

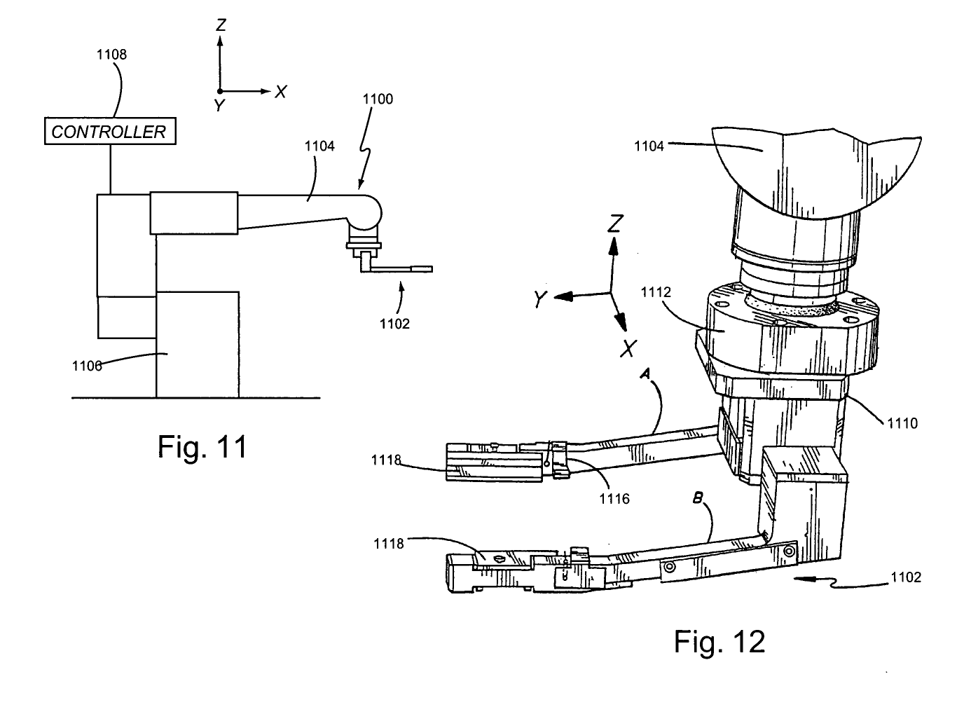

To further illustrate, FIG. 11 schematically depicts robotic gripping component 1100 from a side elevational view according to one embodiment. Robotic gripping component 1100 is an automated robotic device, e.g., for accurately and securely grasping, moving, manipulating and/or positioning containers and other objects. In particular, the design of robotic gripping component 1100 is optionally varied to accommodate different types of objects.

In the embodiment illustrated in FIG. 11, robotic gripping component 1100 includes gripper mechanism 1102 movably connected to boom 1104, which is movable relative to base 1106. Controller 1108, which optionally includes a general purpose computing device, controls the movements of, e.g., gripper mechanism 1102 and boom 1104 in a work perimeter that includes one or more stations that can receive and support selected objects.

Boom 1104 is configured to extend and retract from base 1106. As described above, this defines the work perimeter for robotic gripping component 1100. Work stations for the various other system components are positioned within the work perimeter of boom 1104 as are hand-off areas or other areas that are configured to support or receive objects grasped and moved by gripper mechanism 1102. For example, containers are positioned on a station shelf or container positioning component and can be grasped by gripper mechanism 1102 and moved to another position by boom 1104.

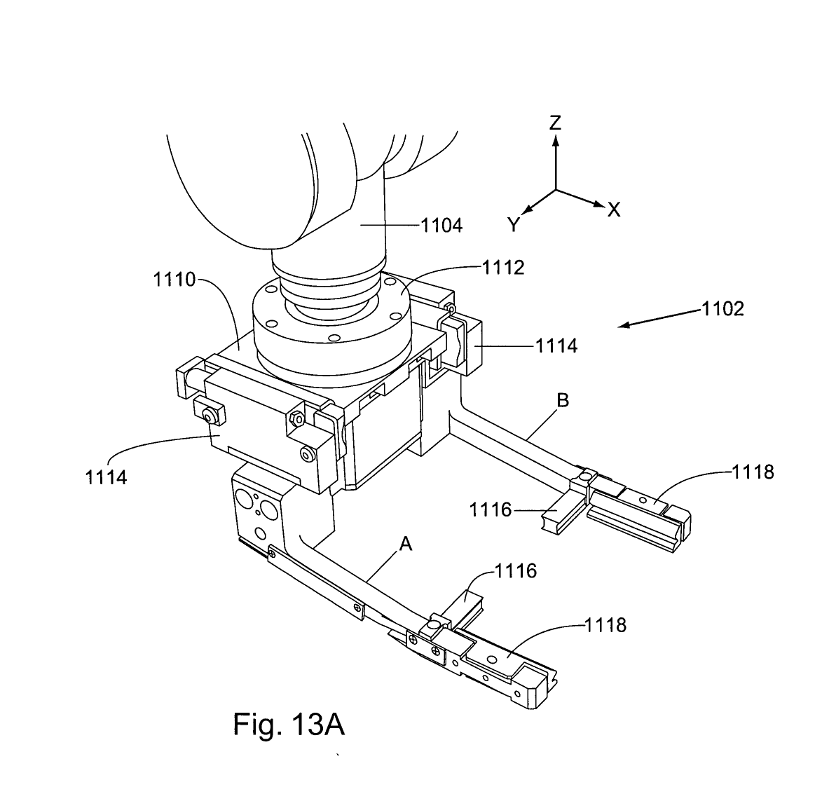

Referring now to FIG. 12, one embodiment of gripper mechanism 1102 is illustrated. Grasping arm A and grasping arm B extend from gripper mechanism body 1110. Although the embodiments described herein include two arms for purposes of clarity of illustration, the gripper mechanisms of the invention optionally include more than two arms, e.g., about three, about four, about five, about six, or more arms. Further, although in certain embodiments, gripper mechanism arms are structured to grasp objects between the arms, other configurations are also optionally included, e.g., such that certain objects can be at least partially, if not entirely, grasped internally, e.g., via one or more cavities disposed in one or more surfaces of the particular objects.

As further shown in FIG. 12, grasping mechanism body 1110 is connected to a deflectable member, such as breakaway 1112, which is deflectably coupled to boom 1104. Breakaway 1112 is typically structured to detect angular, rotational, and compressive forces encountered by gripper mechanism 1102. The breakaway acts as a collision protection device that greatly reduces the possibility of damage to components within the work perimeter by, e.g., the accidental impact of gripper mechanism 1102 or grasping arms A and B with objects. To further illustrate, deflectable members of robotic gripping components generally deflect when the gripper mechanism contacts an object or other item with a force greater than a preset force. The preset force typically includes a torque force and/or a moment force that, e.g., ranges between about 1.0 Newton-meter and about 10.0 Newton-meters. When controller 1108 detects the deflection, it generally stops movement of the robotic gripper mechanism. In one embodiment, breakaway 1112 is a “QuickSTOP™” collision sensor manufactured by Applied Robotics of Glenville, N.Y., U.S.A. Breakaway 1112 is typically a dynamically variable collision sensor that operates, e.g., on an air pressure system. Other types of impact detecting devices are optionally employed, which operate hydraulically, magnetically, or by other means known in the art. In certain embodiments, breakaways are not included in robotic gripping devices used in the systems of the invention. In these embodiments, gripper mechanisms are typically directly coupled to robotic booms.

As also shown, body 1110 connects grasping arms A and B to breakaway 1110. When directed by controller 1108, body 1110 moves grasping arms A and B away from or toward each other, e.g., to grasp and release objects. In one embodiment, body 1110 is manufactured by Robohand of Monroe, Conn., U.S.A. Typically, the grasping arms are pneumatically driven, but other means for operating the arms are also optionally utilized, such as magnetic- and hydraulic-based systems.

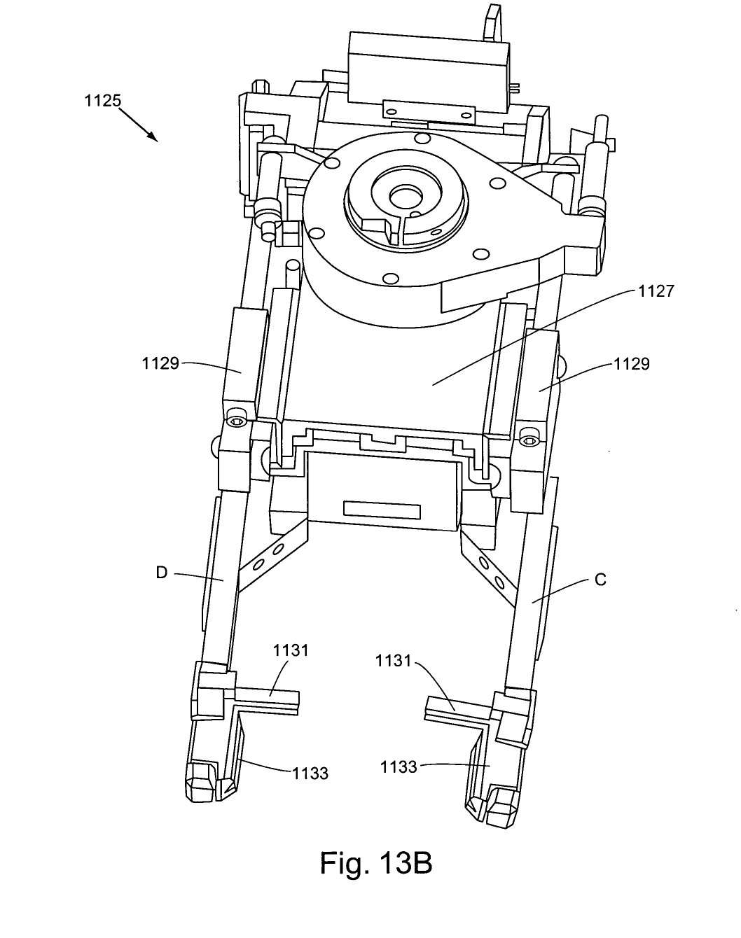

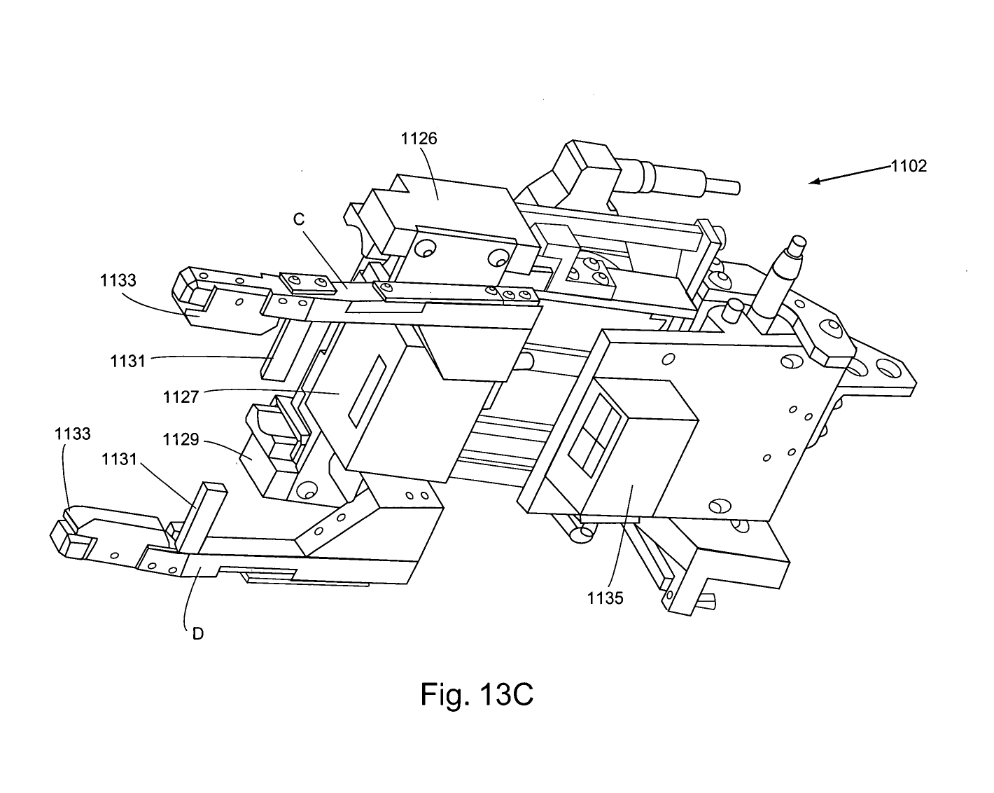

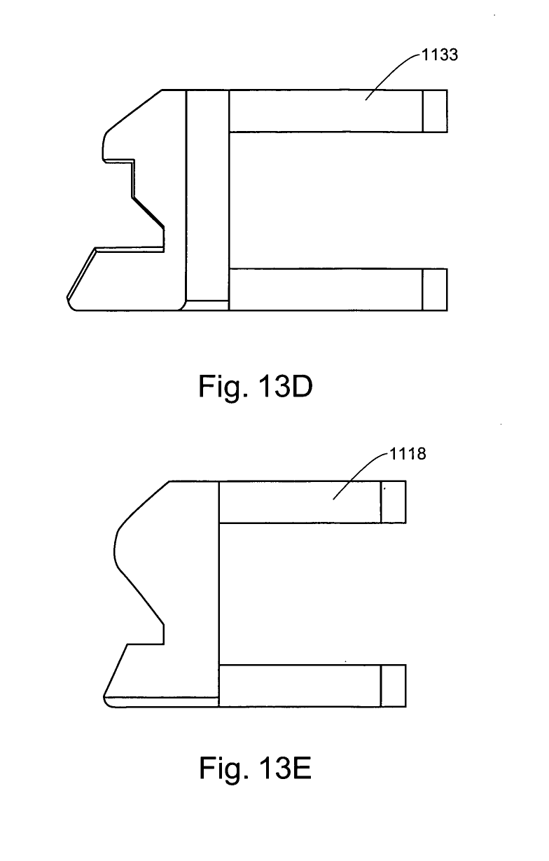

In other embodiments, grasping arms are resiliently coupled to robotic booms such that when an object, such as a multi-well container contacts stops on the grasping arms, the arms reversibly recede from an initial position, e.g., to determine a y-axis position of an object prior to determining the X-axis and Z-axis positions of the object. One of these embodiments is schematically illustrated in FIG. 13A. In particular, FIG. 13A schematically depicts gripper mechanism 1102 that includes arms A and B resiliently coupled to body 1110 via slidable interfaces 1114. Slidable interfaces typically include springs, which resiliently couple, e.g., grasping arms to grasping mechanism bodies. Such resiliency is optionally provided by other interfaces that include, e.g., pneumatic mechanisms, hydraulic mechanisms, or the like. As further shown, arms A and B include stops 1116 and pivot members 1118. As mentioned, the embodiment of gripper mechanism 1102 schematically illustrated in FIG. 13A is optionally used to determine the Y-axis position of an object prior to grasping the object between the arms, that is, prior to determining the X-axis and Z-axis positions of the object. As further shown in FIG. 13A, gripper mechanism 1102 is connected to boom 1104 via breakaway 1112. Breakaways are described in greater detail above.

To further illustrate, FIGS. 13B and C schematically show grasping mechanism 1125 from top and bottom perspective views, respectively, according one embodiment. As shown, grasping mechanism 1125 includes arms C and D resiliently coupled to body 1127 via slidable interfaces 1129 similar to gripper mechanism 1102 described above. As also shown, arms C and D include stops 1131 and pivot members 1133. FIG. 13D schematically shows pivot member 1133 from a front elevational view. Pivot member 1133 is fabricated to accommodate or compensate for various container skirt or rib heights or thicknesses (e.g., about 1 mm, about 1.5 mm, about 2.0 mm, about 2.5 mm, about 3 mm, about 3.5 mm, and/or greater thicknesses) including the skirt heights of, e.g., certain multi-well containers and cell culture containers (e.g., Corning® RoboFlask™ Cell Culture Vessels (Corning, Inc. Life Sciences, Acton, Mass., USA), etc.). Pivot member 1133 can typically accommodate these types of ribs. FIG. 13E schematically illustrates pivot member 1118 from gripper mechanism 1102 from a front elevational view. Grasping mechanism 1125 also includes in-line bar code reader 1135, mounted on a height and angled adjustable mechanism of grasping mechanism 1125. Bar code reader 1135 is configured to read bar codes disposed on containers when bar code reader 1135 is within sufficient proximity to the container, such as when the containers are grasped by arms C and D of grasping mechanism 1125. Bar codes are typically used to track the location of containers in the systems of the invention. Other tracking methods know to persons of skill in the art are also optionally utilized. Although not shown, grasping mechanism 1125 is typically coupled to a boom of a robotic gripping device in the systems described herein.

The robots of the systems described herein are typically used to transport one or more sample containers between locations in the systems. In some embodiments, for example, robots transfer samples disposed in sample containers from one work perimeter to another work perimeter, e.g., via a transfer station. To transfer between adjacent work perimeters, a first robot generally retrieves a sample container, positions the container at a transfer station, and then a second robot from an adjacent work perimeter retrieves the container from the transfer station. Alternatively, robots are configured to directly transfer a sample plate from one robot to another.

4. Material Handling Components

In addition to the material handling components described above, e.g., with respect to the fluid transfer devices of the assaying components of the systems of the invention, other material handling components are also optionally included. In certain embodiments, for example, cells are expanded to selected quantities and pooled performing for compound profiling assays. These pooled cells are then typically dispensed into assay plates or other containers using various dispensing devices. Once these assay plates have been prepared, test compounds or reagents are typically transferred into the assay plates, e.g., using the transfer devices of the assaying components described above. Exemplary material handling components that are optionally adapted to perform reagent or cell culture dispensing, container washing, and/or other material handling functions in the systems of the invention are described in, e.g., U.S. Provisional Patent Application No. 60/577,849, entitled “DISPENSING SYSTEMS, SOFTWARE, AND RELATED METHODS,” filed Jun. 7, 2004 by Chang et al., U.S. Provisional Patent Application No. 60/598,994, entitled “MULTI-WELL CONTAINER PROCESSING SYSTEMS, SYSTEM COMPONENTS, AND RELATED METHODS,” filed Aug. 4, 2004 by Micklash II et al., International Publication No. WO 2004/091746, entitled “MATERIAL REMOVAL AND DISPENSING DEVICES, SYSTEMS, AND METHODS,” filed Apr. 7, 2004 by Micklash II et al., U.S. patent application Ser. No. 11/003,026, entitled “MATERIAL CONVEYING SYSTEMS, COMPUTER PROGRAM PRODUCTS, AND METHODS,” filed Dec. 1, 2004 by Chang et al., U.S. Patent Publication No. US-2003/0175164, entitled “DEVICES, SYSTEMS, AND METHODS OF MANIFOLDING MATERIALS,” filed Sep. 18, 2003 by Micklash II et al., U.S. Pat. No. 6,659,142, entitled “APPARATUS AND METHODS FOR PREPARING FLUID MIXTURES,” to Downs et al., and U.S. Pat. No. 6,827,113, entitled “MASSIVELY PARALLEL FLUID DISPENSING SYSTEMS AND METHODS,” filed Mar. 27, 2002 by Downs et al., which are each incorporated by reference. In addition, exemplary micro-well plate stations that are optionally adapted for use in the systems of the invention are also described in, e.g., Reidel et al. (2005) “Low Temperature Microplate Stations,” JALA 10:29-34, which is incorporated by reference.

Other automated devices that are optionally used in the systems of the invention are replating stations positioned at station locations in one or more work perimeters. These devices are typically used to replate or replicate a plurality of samples from one or more small sample plates into a single large sample plate. For example, compounds are optionally transferred or replated from 96 well to 384 well microtiter plates and/or from 384 to 1536-well plates. These stations generally use visual and readable controls to track the reformatting and allow the user to verify that the reformatting was successful. A Tecan Miniprep robotic station (Tecan US, Durham, N.C., USA), which comprises an automatic sample processor, is one example of a device that is suitable for replating operations.

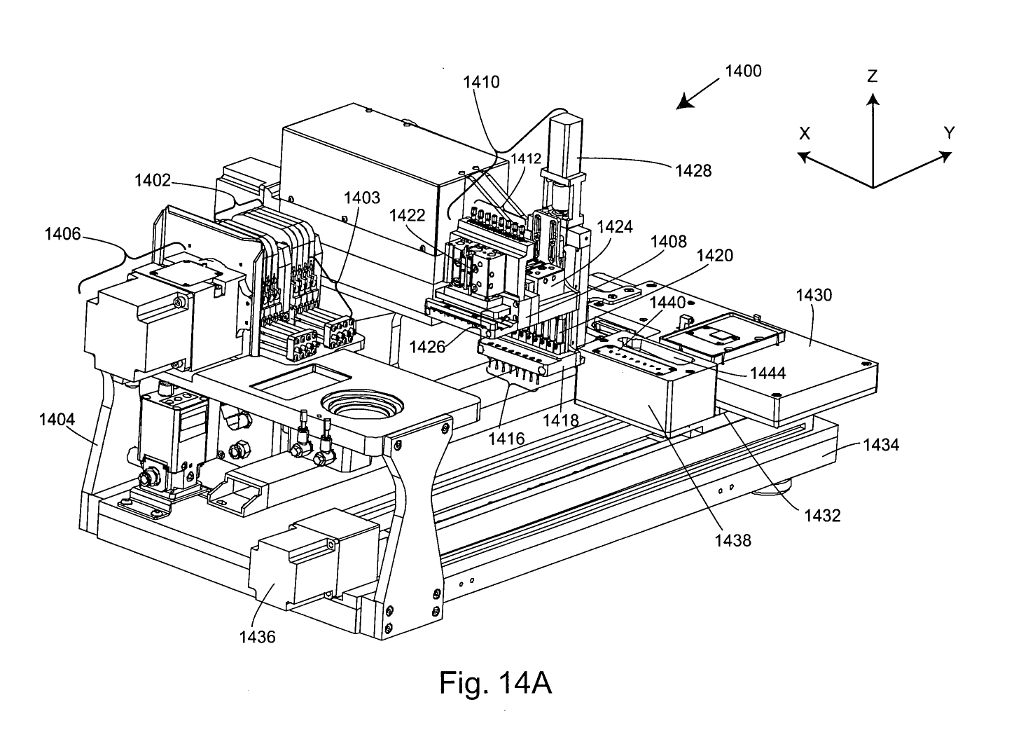

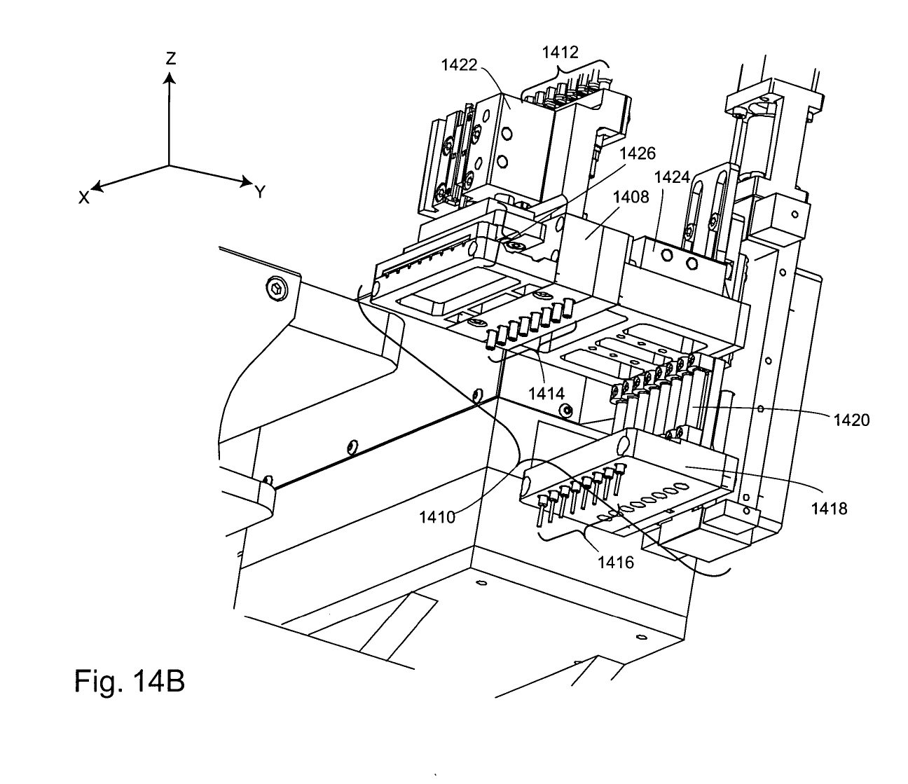

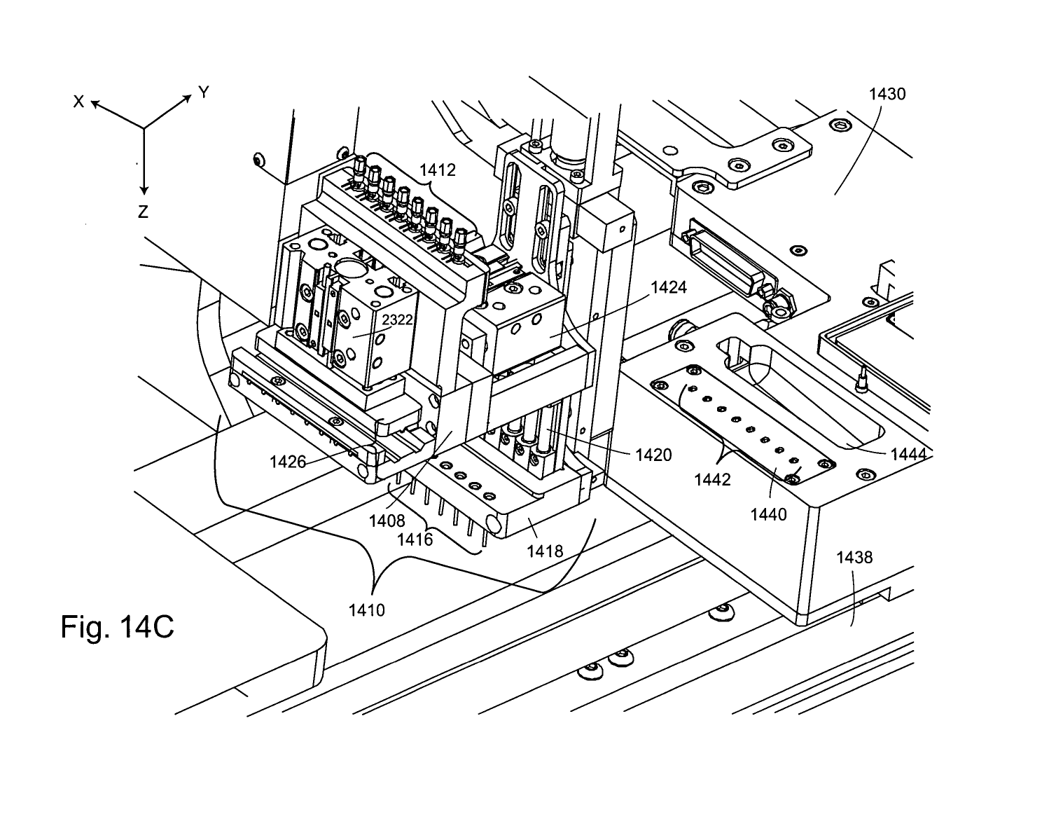

To further illustrate additional material handling components that are optionally included as components of the systems of the invention, FIGS. 14A-C schematically depict dispensing station 1400 according to one embodiment. As shown, dispensing station 1400 includes peristaltic pump 1402 (e.g., a multi-channel low volume peristaltic pump) mounted on mounting component 1404 (shown as a rigid frame). Dispensing station 1400 also includes a feedback component that comprises drive motor 1406, which typically includes a position encoder and gear reduction, and which is operably connected to peristaltic pump 1402 to effect precisely controlled rotation of the rotatable roller support of peristaltic pump 1402. The feedback component also includes a control system for drive motor 1406 (not shown in FIG. 14) that is capable of position feedback control.

During operation, conduits (not shown in FIG. 14) are generally disposed between the compression surfaces and rollers of peristaltic pump 1402. In addition, one set of termini of the conduits fluidly communicate with the same or different material sources (not shown in FIG. 14), while the other set of termini are operably connected to and fluidly communicate with fluid junction block 1408 of dispensing component 1410. As also shown, dispensing station 1400 includes tube stretchers 1403, which are designed to give the user fine adjustment over the flow rate of each peristaltic channel. More specifically, tube stretchers 1403 mechanically increase the length of associated peristaltic tubing or conduits. As the length of a given tube is increased, the inner diameter of that tube decreases and the volume conveyed per pulse or rotational increment is also decreased. This gives the user a fine adjustment to the flow rate for each peristaltic channel. In some embodiments, further adjustments can be made by varying the spacing between peristaltic pump cartridges and rollers.

FIGS. 14B and C schematically illustrate detailed bottom and top perspective views, respectively, of dispensing component 1410 from dispensing station 1400. Solenoid valves 1412 fluidly communicate with the same or different pressure sources (not within view) (e.g., a pressurized gas source, a pressurized second fluidic material source, a pump, etc.) and with fluid junction block 1408 via conduits (not shown in FIG. 14). Outlets 1414 of fluid junction block 1408 fluidly communicate with dispensing tips 1416 disposed in dispense head 1418 via conduits (not shown in FIG. 14), which conduits form conduit coils disposed around vertically mounted posts. As also shown, dispensing component 1410 also includes air tables 1422 and 1424. Air table 1422 effects operation of pinch valve 1426, whereas 1424 is operably connected to a gas valve (not within view) of fluid junction block 1408 to regulate the flow of gas into fluid junction block 1408 to introduce gaseous gaps to prevent fluid mixing.

In addition, dispensing component 1410 of dispensing station 1400 also includes Z-axis linear motion component 1428 (e.g., a compact, high speed, short travel Z-axis motion component or system), which is a positioning component that effects Z-axis translation of dispensing tips 1416 relative, e.g., multi-well plates, membranes, etc. disposed on object holder or container positioning component 1430. Container positioning component 1430 is operably connected to X/Y-axis linear motion components 1432 (shown as tables), which move object holder 1430 relative to dispensing tips 1416 along the X- and Y-axes. X/Y-axis linear motion components 1432 are also mounted on support element 1434, which forms part of mounting component 1404. One or more motors (e.g., solenoid motors, etc.) are generally operably connected to these dispensing stations to effect motion of object holders on X/Y-axis linear motion tables. For example, solenoid motor 1436 effects motion of object holder 1430 in dispensing station 1400. Although not within view in FIGS. 14A-C, dispensing station 1400 also generally includes control drives, e.g., for X/Y-axis linear motion components 1432 and position feedback for drive motor 1406. As also shown, cleaning component 1438, which is used to clean dispensing tips 1416 is also included. In particular, cleaning component 1438 includes vacuum chamber 1440 having orifices 1442 that correspond to dispensing tips 1416 such that when dispensing tips 1416 are disposed proximal to orifices 1442 under a vacuum applied by vacuum chamber 1440, adherent material is removed at least from external surfaces of dispensing tips 1416. Cleaning component 1438 also includes fluid container 1444 disposed next to vacuum chamber 1440. In certain embodiments, fluid container 1444 contains a cleaning solvent into which dispensing tips 1416 can be lowered by Z-axis linear motion component 1428, e.g., prior to applying a vacuum to dispensing tips 1416 at vacuum chamber 1440. Optionally, fluid container 1444 is used as a waste collection component.

The dispensing stations of the systems of the invention also typically include controllers (also not shown in FIG. 14) that are configured to effect rotation of peristaltic pump roller supports in selected rotational increments, to effect application of pressure from pressure sources, to effect motion of linear motion components, and/or the like.

5. Incubation, Refrigeration, and Container Storage Components

The systems of the invention optionally include various incubation, refrigeration, and storage stations that are within a work perimeter of, and accessible by, a given rotational robot or other robotic gripping device, e.g., at selected station locations. In certain embodiments, for example, incubation stations are used to culture cell populations, e.g., as part of an expansion or growth process prior to using the cells in a compound profiling process. In addition, as cell cultures are split using cell culture passaging stations, sample aliquots are typically automatically removed from cell culture flasks at selected intervals and archived in freezer stations included in the systems of the invention. To further illustrate, compound and assay multi-well containers are also typically stored at least transiently in incubation, refrigeration, and other storage stations, e.g., prior to being utilized to perform a given assay in an assaying component of the system. Exemplary incubation and other storage devices that are optionally adapted for use in the systems of the invention are also described in, e.g., U.S. patent application Ser. No. 11/140,530, entitled “HIGH THROUGHPUT INCUBATION DEVICES AND SYSTEMS,” filed May 27, 2005 by Shaw et al., International Publication No. WO 03/008103, entitled “HIGH THROUGHPUT INCUBATION DEVICES,” filed Jul. 18, 2002 by Weselak et al., U.S. Patent Publication No. 2004/0236463, entitled “COMPOUND STORAGE SYSTEM,” filed Feb. 6, 2004 by Weselak et al., and U.S. Provisional Patent Application No. 60/598,929, entitled “OBJECT STORAGE DEVICES, SYSTEMS, AND RELATED METHODS,” filed Aug. 4, 2004 by Shaw et al., which are each incorporated by reference.

To further illustrate, incubation devices utilized in the systems of the invention typically include a housing with a plurality of doors disposed in, e.g., an access panel located on a side of the device. Typically, a robotic gripping device located outside the incubation device is used to open individual doors located in the access panel as it loads or unloads containers (e.g., multi-well containers, cell culture flasks, etc) into or out of the incubation device. This generally reduces the air exchange between the external environment and the internal environment of the incubation device along with limiting the moving parts within the interior of the incubation device. As a result, the incubation devices used in the systems of the invention provide a controlled environment for maintaining parameters, such as humidity, temperature, gas conditions (e.g., CO₂, N₂, or other gas levels).

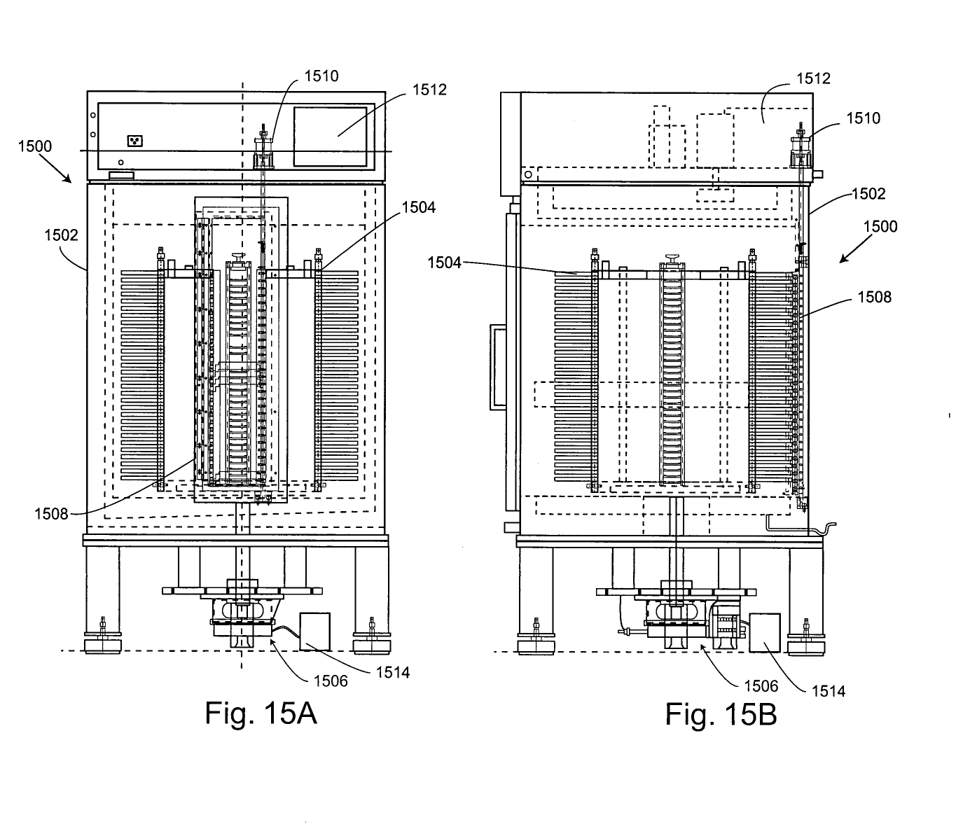

One embodiment of an incubation device is illustrated schematically in FIG. 15. In particular, FIG. 15A schematically depicts a front cutaway view of incubation device 1500. As shown, incubation device 1500 includes housing 1502 having carrousel with vertical columns of shelves 1504 disposed in housing 1502. Rotational mechanism 1506 (shown as an external motor) is operably connected to carrousel 1504 to rotate selected vertical columns of carrousel 1504 into alignment with vertical column of doors 1508. In certain embodiments, rotational mechanisms are configured to rotate the rotatable carrousels in one or more selectable modes. To illustrate, one exemplary selectable mode includes an oscillation (e.g., a side-to-side motion, etc.) of rotatable carrousels as the rotatable carrousels are rotated, e.g., to agitate containers or other objects disposed on the shelves of the carrousels. Typically, controller 1514 controls rotation of carrousel 1504 via rotational mechanism 1506, e.g., in these selectable modes. Incubation device 1500 also includes controller 1512, which controls one or more internal housing conditions. FIG. 15A also schematically illustrates door hold-open mechanism 1510 that includes a member (e.g., a rod, a column, a pole, a slat, a bar and the like) having a plurality of prongs (or a series of pins or other stops) for holding accessed doors of vertical column of doors 1508 open. FIG. 15B schematically depicts incubation device 1500 from a side cutaway view.

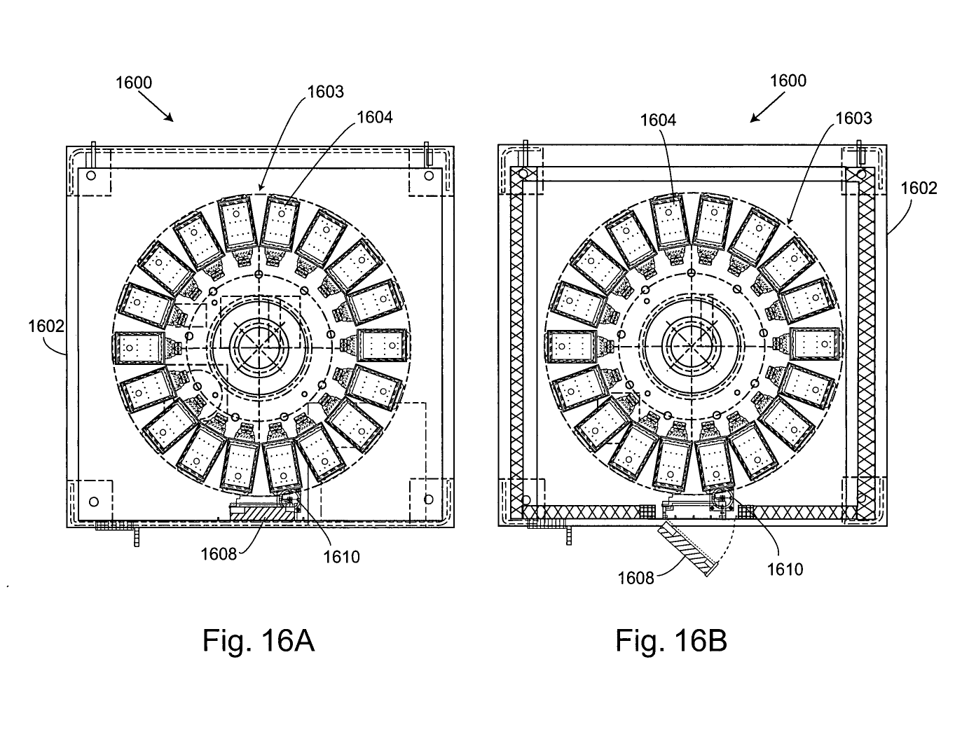

As referred to above, a rotating vertical carrousel with multiple columns (commonly referred to as “hotels”) and multiple shelves is typically located inside the incubation devices. To further illustrate, FIG. 16A schematically depicts a top cutaway view of incubation device 1600, while FIG. 16B schematically depicts a bottom cutaway view of incubation device 1600 according to one embodiment. Incubation device 1600 includes carrousel 1603 with a plurality of shelves 1604 disposed in housing 1602. A rotational mechanism (not shown) is operably connected to carrousel 1603 to rotate selected vertical columns of carrousel 1603 (e.g., about a Z-axis) into alignment with vertical column of doors 1608. Incubation device 1600 also includes door hold-open mechanism 1610 that includes a member (e.g., a rod, a column, a pole, a slat, a bar and the like) having a plurality of stops (shown as prongs) for holding accessed doors of vertical column of doors 1608 open. Vertical column of doors 1608 is hinged to housing 1602, which provides the ability to open or close vertical column of doors 1608. FIG. 16A schematically depicts vertical column of doors 1608 in a closed position, while FIG. 16B schematically depicts vertical column of doors 1608 in an open position.

As referred to above, the incubation devices of system of the invention optionally include access panels (e.g., vertical access panels, horizontal access panels, etc.), which are typically located on the sides of the devices. In some embodiments, access panels are attached to device housings via hinges. An open access panel provides access to a plurality of shelves in a carrousel and the interior compartment of the particular incubation device. Optionally, the access panel includes a gasket to further seal the interior environment of the given incubation device from the exterior environment and a lock, latch, and/or other mechanism to maintain the access panel in a closed position when desired.

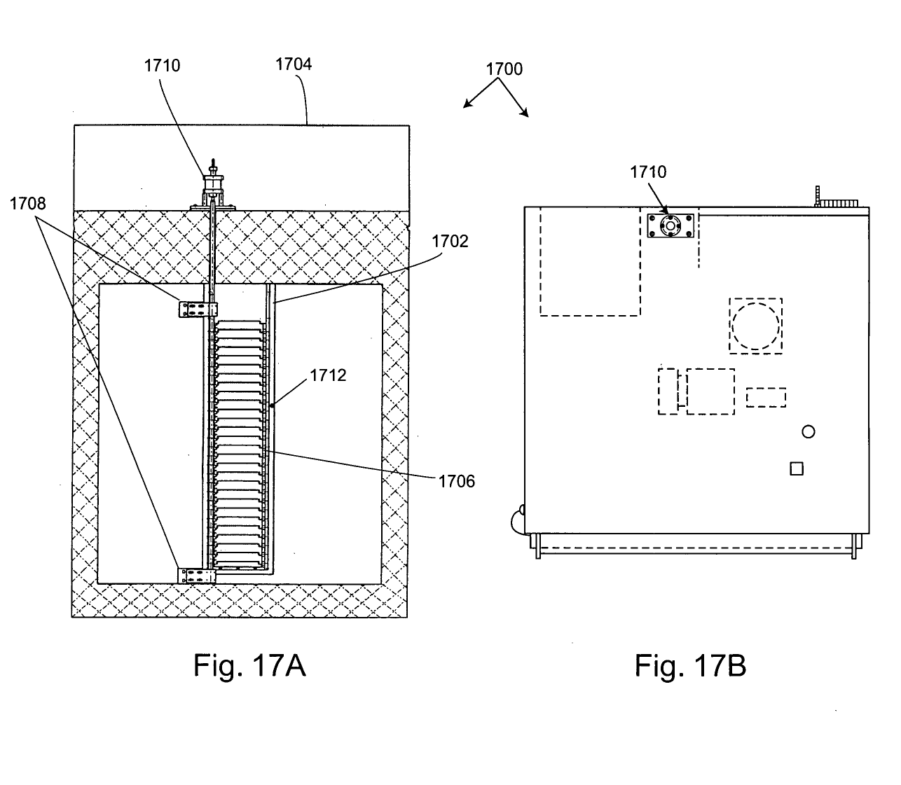

FIG. 17A schematically depicts a front view of incubation device 1700 according to one embodiment. As shown, access panel 1702 is disposed in a surface of device housing 1704. Access panel 1702 includes vertical column of doors 1706 and is attached to device housing 1704 by hinges 1708. A portion of door hold-open mechanism 1710 is also illustrated. FIG. 17B schematically depicts a top view of incubation device 1700.

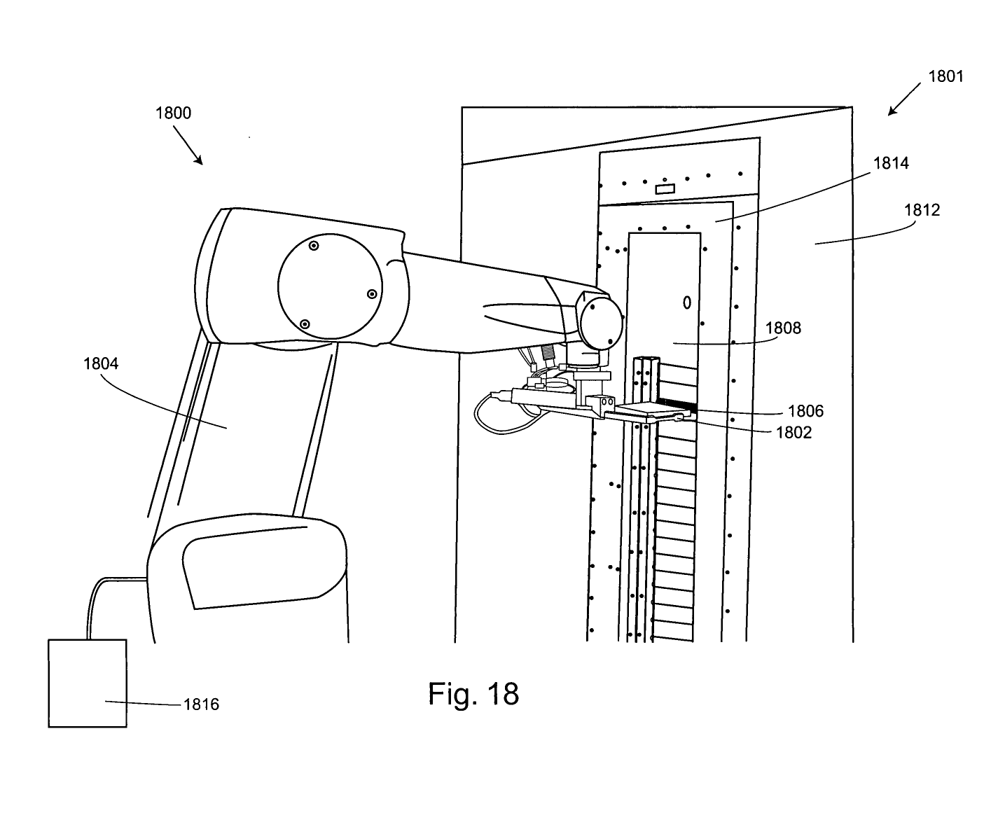

Individual actuators are typically not needed to open doors because a robotic gripping device typically provides mechanical actuation to open selected doors. Thus, incubation devices need not have any internal mechanism for opening the doors in, e.g., a given vertical column or horizontal row of doors. Since only relatively small doors are open at a time, air exchange between the interior of an incubation device and the outside atmosphere is reduced. FIG. 18 depicts robotic gripping device 1800 (e.g., a rotational robot) located outside incubation device 1801 opening door 1806 on vertical access panel 1814. Robotic gripping device 1800 loads and unloads containers into and out of incubation device 1801. More specifically, FIG. 18 schematically depicts gripper mechanism 1802 of robotic gripping device 1804 interfacing with door 1806 in vertical column of doors 1808 of housing 1812 in this exemplary embodiment. Robotic gripping device 1800 also includes logical device 1816 for controlling movement of robotic armature 1804. Robotic gripping devices are also described above.

The systems of the invention optionally include other storage devices, including certain modular object storage devices. These devices can be used, e.g., to store and manage large numbers of objects, such as compound libraries stored in multi-well containers. Robotic gripping devices are generally configured to translocate multi-well plates, substrates, cell culture flasks, or the like to and/or from object storage module shelves, and/or object storage modules to and/or from object storage module receiving areas of support elements of these modular object storage devices. As described above, system components such as these are optionally housed within enclosures or chambers, e.g., to prevent the contamination of objects stored on the shelves of modular object storage devices.

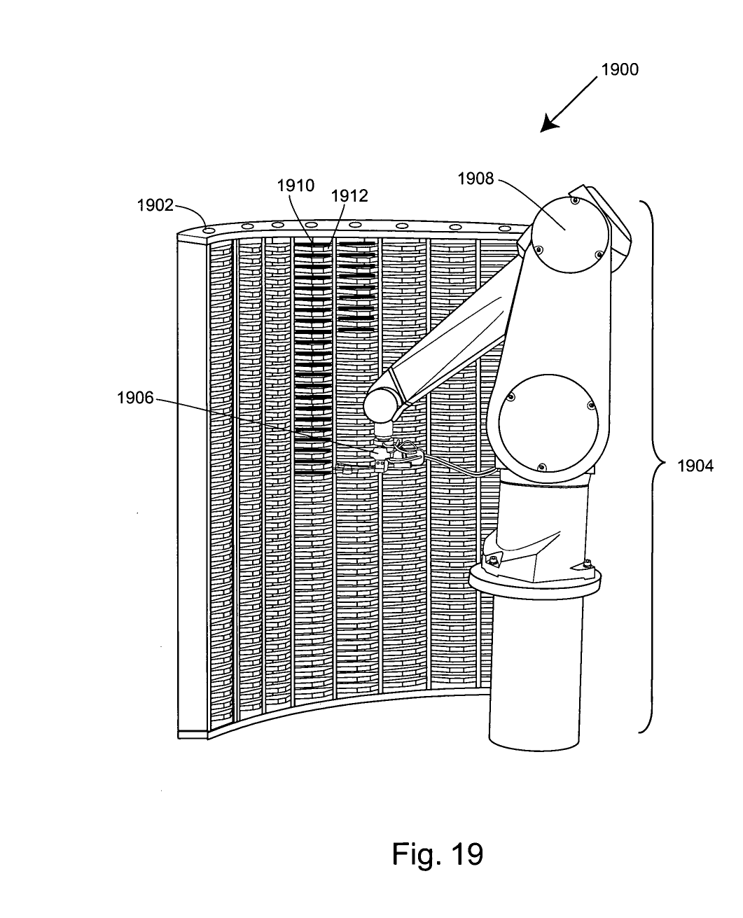

To illustrate, FIG. 19 schematically illustrates container storage station 1900, which includes modular object storage device 1902 and robotic gripping device 1904 from a perspective view. As shown, robotic gripping device 1904 includes gripper mechanism 1906 operably connected to robotic armature or boom 1908, which positions gripper mechanism 1906 relative to multi-well plates 1910 such that multi-well plates 1910 can be grasped by gripper mechanism 1906 and translocated to and/or from shelves 1912 of modular object storage device 1902 by boom 1908. Typically, robotic gripping device 1904 translocates multi-well plates 1910 between modular object storage device 1902 and another system component, such as a dispensing station, an assaying component, or other work station, e.g., for processing or analysis.

6. Lid Processing Devices

To reduce contamination and evaporative effects, it is sometimes desirable to provide sample containers with lids. A lid that sufficiently seals a given container, such as a multi-well container not only reduces evaporation and contamination, but also generally allows gases to diffuse into sample wells more consistently and reliably. Lids typically have a gripping structure, such as a gripping edge, that a robotic gripping device engages when adding or removing the lids from the containers. For example, U.S. Pat. No. 6,534,014, entitled “SPECIMEN PLATE LID AND METHOD OF USING,” filed May 11, 2000 by Mainquist et al., which is incorporated by reference, discloses specimen plate lids for robotic use that are optionally utilized to seal containers in the systems described herein. Further, lid processing devices or stations are also optionally included as components of the systems described herein, e.g., for adding and removing lids to and from containers.

I . Containers

The automated cellular disruption systems of the invention can be adapted to disrupt cells disposed in a wide variety of containers. Exemplary containers that are optionally utilized include various single- or multi-well containers, such as petri dishes, beakers, flasks, vials, test tubes, and micro-well or microtiter plates (e.g., microplates meeting the SBS-ANSI standards, etc.), among others known to persons of skill in the art. Certain standard multi-well containers include, e.g., 6, 12, 24, 48, 96, 192, 384, 768, 1536, or more wells, and are generally available from various commercial suppliers including, e.g., Greiner Bio-One International AG (Frickenhausen, Germany), Nalge Nunc International (Rochester, N.Y., USA), H+P Labortechnik AG (Oberschleiβheim, Germany), and the like. To illustrate, a representative microtiter plate is schematically illustrated in, e.g., FIGS. 9A-C.

In some embodiments, containers are labeled with at least one identifier, for example, a bar code, RF tag, color code, or other label. To illustrate, when containers are labeled with bar codes, robotic gripping components, which translocate containers in certain system embodiments, typically include bar code readers. The bar code readers are optionally positioned on the robotic arms or any other position on the robot depending upon the application and type of container used. In some embodiments, bar code readers are positioned at stations that are separate from robotic gripping components. By identifying each container with a bar code, RF tag, or color code, a system can positively identify each container, e.g., when retrieving, processing, or detecting properties of samples in the containers. In addition, the information is also optionally used to provide reports regarding assay outcomes and results, and to provide an inventory of a large number of samples, e.g. libraries of nucleic acid samples. For example, an inventory is optionally used to compare a list of desired plates with a list of plates present in the system, and notify an operator of any discrepancies.

In certain embodiments, when a multi-well container is provided with a bar code at opposite ends, and the bar codes have indicia relating orientation, the systems of the present invention determine which end of the container is facing the robotic gripping component. For example, one end of the container optionally has a bar code with an even code, while the opposite end of the container has an odd numbered code. Accordingly, the robotic gripping components used in certain systems of the invention easily determine whether a leading or trailing edge of a container is facing the bar code reader in the robotic gripping components. In this manner, robotic gripping components reliably and consistently determine which end of a container to insert into or onto a container positioning component, an incubation component, a container storage component, etc.

J. System Component Fabrication

System components (e.g., cellular disruption components, holding blocks, cellular disruption implements, container positioning components, housings, shelves, support elements, frame components, etc.) or portions thereof are optionally formed by various fabrication techniques or combinations of such techniques including, e.g., milling, machining, welding, stamping, engraving, injection molding, cast molding, embossing, extrusion, etching (e.g., electrochemical etching, etc.), or other techniques. These and other suitable fabrication techniques are generally known in the art and described in, e.g., Altintas, Manufacturing Automation: Metal Cutting Mechanics, Machine Tool Vibrations, and CNC Design, Cambridge University Press (2000), Molinari et al. (Eds.), Metal Cutting and High Speed Machining, Kluwer Academic Publishers (2002), Stephenson et al., Metal Cutting Theory and Practice, Marcel Dekker (1997), Rosato, Injection Molding Handbook, 3^{rd} Ed., Kluwer Academic Publishers (2000), Fundamentals of Injection Molding, W. J. T. Associates (2000), Whelan, Injection Molding of Thermoplastics Materials, Vol. 2, Chapman & Hall (1991), Fisher, Extrusion of Plastics, Halsted Press (1976), and Chung, Extrusion of Polymers: Theory and Practice, Hanser-Gardner Publications (2000), which are each incorporated by reference. In certain embodiments, following fabrication, device components or portions thereof are optionally further processed, e.g., by coating surfaces with a hydrophilic coating, a hydrophobic coating (e.g., a Xylan 1010DF/870 Black coating available from Whitford Corporation (West Chester, Pa., USA), epoxy powder coatings available from DuPont Powder Coatings USA, Inc. (Houston, Tex., USA)), or the like, e.g., to prevent interactions between component surfaces and reagents, samples, or the like, to provide a desired appearance, and/or the like.

The systems of the invention are typically assembled from individually fabricated component parts (e.g., shelves, housings, frame components, etc). Component fabrication materials are generally selected according to properties, such as durability, expense, or the like. In certain embodiments, components or portions thereof are fabricated from various metallic materials, such as stainless steel, anodized aluminum, or the like. Optionally, system components are fabricated at least in part from polymeric materials such as, polytetrafluoroethylene (TEFLON™), polypropylene, polystyrene, polysulfone, polyethylene, polymethylpentene, polydimethylsiloxane (PDMS), polycarbonate, polyvinylchloride (PVC), polymethylmethacrylate (PMMA), or the like. Component parts are also optionally fabricated from other materials including, e.g., wood, glass, silicon, or the like. In addition, certain component parts are typically assembled using various attachment methods, e.g., welding, bonding, adhering, bolting, riveting, etc.

### IV. CELLULAR ASSAYING METHODS

The systems of the invention can be used or adapted for use in performing a wide variety of cell-based assaying methods, including cell motility screens, viability assays, etc. Typically, these methods include culturing or otherwise providing the cells of interest (e.g., mammalian cells, etc.) on surfaces of containers, such as on the bottom walls of microtiter plate wells (e.g., as confluent monolayers). One exemplary source for many different cell lines (including normal and diseased cell lines), which may be of use in performing these methods, is the American Type Culture Collection (ATCC) (Manassas, Va., USA). In addition, many different cell culturing techniques, which are optionally utilized in performing the methods of the invention, are generally known to persons of skill in the art. Some of these as well as various cell culturing systems components that can be utilized are also described in, e.g., Freshney, Culture of Animal Cells: A Manual of Basic Technique, 4^{th} Ed., Wiley-Liss (2000), U.S. Provisional Patent Application No. 60/664,640, entitled “COMPOUND PROFILING DEVICES, SYSTEMS, AND RELATED METHODS”, filed Mar. 22, 2005 by Chang et al., and U.S. Provisional Patent Application No. 60/680,132, entitled “COMPOUND PROFILING DEVICES, SYSTEMS, AND RELATED METHODS”, filed May 11, 2005 by Chang et al., which are each incorporated by reference. These methods also generally include positioning (e.g., manually or robotically) the containers on the container positioning components of the systems described herein, and moving the cellular disruption components and/or the containers in accordance with user selected substantially uniform modes as described herein such that the cellular disruption implements being utilized in the particular system disrupt (e.g., scratch, wound, etc.) the cells in the containers. An example of a genetic screen for modulators of cancer cell motility is provided below.

The assaying methods of the invention generally include contacting the cells with, or introducing into the cells (e.g., via electroporation, transfection, etc.) modulators or candidate modulators prior to, during, and/or after the cells are disrupted. Exemplary modulators or candidate modulators include inorganic molecules, organic molecules, vectors (e.g., nucleic acid vectors, such as plasmids, cosmids, artificial chromosomes, etc.) comprising or encoding the modulators or the candidate modulators, sense nucleic acids, anti-sense nucleic acids, transcription factors, complementary DNAs (cDNAs), short interfering RNAs (siRNAs), microRNAs (miRNAs), synthetic hairpin RNAs (shRNAs), and the like. The methodology of RNA interference (RNAi), for example, is also described in, e.g., Sandy et al. (2005) “Mammalian RNAi: a practical guide,” Biotechniques 39(2):215-224 and Fitzgerald (2005) “RNAi versus small molecules: different mechanisms and specificities can lead to different outcomes,” Curr Opin Drug Discov Devel. 8(5):557-566, which are both incorporated by reference.

The methods of the invention also typically include detecting one or more detectable properties of the cells prior to, during, and/or after the cells are disrupted. For example, this optionally includes imaging the cells using an automated fluorescent microscope (e.g., available from Q3DM, Inc (Beckman Coulter, San Diego, Calif., USA)) or another image capturing device or detection component. Exemplary detectable properties include a presence, absence, or extent of cellular motility. The methods also generally include correlating these detected detectable properties with particular genes of the cells, and/or comparing the detected detectable properties with suitable controls.

### V. EXAMPLES

It is understood that these examples and embodiments described herein are for illustrative purposes only and are not intended to limit the scope of the claimed invention. It is also understood that various modifications or changes in light the examples and embodiments described herein will be suggested to persons skilled in the art and are to be included within the spirit and purview of this application and scope of the appended claims.

A. Genetic Screen for Modulators of Tumor Cell Motility

1. Overview

Tumor cells become metastatic through the acquisition of traits that allow them to disseminate, re-localize, and colonize/grow in organs distant from their site of origin. The invasive potential of a tumor cell can, in part, be measured by their ability to migrate across a “wound”—a simple scratch in a confluent layer of cells in culture dishes. This potential to migrate is often correlated with a cells' ability to penetrate and migrate through a matrix (e.g. matrigel, collagen, etc). This example describes a representative genetic screen designed to identify genes involved in promoting tumor cell metastasis. The screen utilizes an exemplary high precision 384-well-based cellular disruption system of the invention coupled with automated microscopy. As noted below, the automated assay described in this example is also adaptable to, e.g., small molecule and cDNA gain-of-function screens and thus can provide insight into the movement of cells in many different contexts.

In overview, the exemplary high precision 384-well-based cellular disruption system used a set of 384 12.5 μl pipette tips to scratch confluent cells on the base of a 384-well plate. Tumor cells with migratory potential were plated at high density in 384-well plates in which different siRNAs (or cDNAs) had been pre-plated. For siRNAs, the cells were incubated for 48 hours, scratched, and incubated for a further 12 hours to allow cells to migrate. For assessing the effect of small molecules, cells were plated, grown to confluency, and molecules were added 12 hours prior to scratching. Following scratching, cells were incubated for 12 hours as above. Following the timed post-scratch incubation, cells were fixed with formaldehyde and stained with the nuclear stain DAPI. Each well of the 384-well plate was then photographed by the Q3DM high content imaging microscope using a 4× objective to visualize a majority of the space of each well. All assays were conducted in duplicate to assess the reproducibility of the results.

It was possible that a lack of closure of the wound/scratch was due to a loss of cell viability, leading to the appearance of a specific block in cellular motility. To control for this possibility, a cell viability assay was run in parallel to the scratch assay. The viability plates were processed identically to sister scratch assay plates up until the point of fixation. At this point, the plates destined for the viability assay were incubated with Cell Titre Glo (Promega, Madison, Wis., USA), a reagent that measures cell viability through the measurement of ATP metabolism. Following incubation, the luminescent intensities of the wells containing Cell Titre Glo were recorded, with the intensity being proportional to the number of viable metabolizing cells in the well.

2. Assay Hardware

The automated 384-well plate-based cellular disruption system used in the screen was fabricated as described herein. To illustrate, a system that is similar to the one referred to in this example is schematically depicted in FIG. 1, which is described further above. Briefly, the system included a machined aluminum holding block into which 384 orifices had been drilled wide enough to accommodate 12.5 μl pipette tips (Matrix Technologies, Hudson, N.H., USA). Sterilized pipette tips were inserted into the holding block, which was placed in a holding block receiving area of the system to suspend the holding block on a vertical tracking arm of a translational mechanism of the system. The holding block was raised against a top plate or cellular disruption implement locating component to stabilize the tip positions and prevent movement upon contacting the tips with the plates. 384-well clear bottom tissue culture plates (Greiner Bio-One, Frickenhausen, Germany) were placed on a level platform or container positioning component below the aluminum block. Once the equipment was initiated, the aluminum holding block was automatically lowered to a point at which the pipette tips touched the bottom of each of the 384 wells. With the pipette tip holding block engaged, the container positioning component was shifted about 3 mm (well diameter=3.70 mm) by hydraulic pressure, resulting in uniform cellular disruption or “scratches” in each of the 384 wells. Following scratching, the holding block was raised up from the plate, and the container positioning component returned to the start position to allow plates to be manually switched by the user.

3. Other Materials and Methods

siRNAs: Small interfering (si)RNAs were purchased from Dharmacon (Lafayette, Colo., USA) or Qiagen (Valencia, Calif., USA), prepared and dispensed into 384-well plates as described (Aza-Blanc et al. (2003) Mol. Cell. 12:627-637, which is incorporated by reference). The library is comprised of 10,996 siRNAs targeting 5,234 unique genes. Approximately 500 siRNAs in the collection are targeted to known and predicted human kinases as described; the remaining 10,500 siRNAs were designed to target specific families of genes which are considered pharmaceutically tractable, such as proteases, G-protein coupled receptors (GPCRs), cytokines and cytokine receptors, as well as other classes of genes, such as transcription factors, components of the cell cycle and apoptotic machinery.

384-well scratch assay: Cells were plated at high density (4,000-5,000 cells per well) in media supplemented with 10% FBS. Cell density was calculated to result in >95% confluence at the time of scratching, accounting for the toxicity of the transfection reagent lipofectamine 2000 (Invitrogen Corp., Carlsbad, Calif., USA). Cells were added to a siRNA/transfection reagent cocktail and deposited on the pre-plated siRNAs, resulting in reverse transfection, as described previously (Aza-Blanc et al. (2003) Mol. Cell. 12:627-637, which is incorporated by reference). For small molecule experiments, compounds were added 12 hours prior to scratching at a final concentration of 0.5% DMSO. Media was changed in all experiments 24 hours after plating. Assay plates were fitted with metal low-evaporation covers and incubated at 37° C., 5% CO₂ in humidified tissue culture incubators. All liquid dispensing steps were performed using a Multidrop 384-well dispenser (Titertek, Huntsville, Ala., USA). At 48 hours, confluent monolayers were scratched as described above. Cells were allowed to traverse the wound, typically resulting in closure of control cell wells by 12 hours. Following wound closure, cells were fixed with formaldehyde (Sigma, St. Louis, Mo., USA) at a final concentration of 3.7% for 1 hour, washed and stained with the nuclear stain, DAPI (Molecular Probes, Eugene, Oreg., USA). Each well of the 384-well plate was photographed by a fluorescent microscope re-tooled by Q3DM Inc (Beckman Coulter, San Diego, Calif., USA) to automate image capture. A 4× objective lens was used to capture a majority of the space within each well. Images were collated and quantitatively scored as described below. For display purposes, images were imported into ImageJ (downloaded from the NIH; http://rsb.info.nih.gov/ij/). DAPI-stained nuclei were encircled and the images inverted.

Cell viability: Cells were plated into a “sister” set of 384-well siRNA assay plates and processed identically to the scratch plates. Viability was measured using Cell Titre Glo (Promega, Madison, Wis., USA). The mean luminescent intensity of each plate was calculated, and the percent of the plate mean was calculated for each well. Small interfering RNAs or compounds resulting in an average percent mean of less than 90% were considered to negatively impact viability, and were eliminated from further study.

Quantitative scoring method: Automated microscopic capture of the assay generated one grayscale image per well (4× magnification). Bright regions represented DAPI-stained nuclei (cells) and black regions represented background; pixel intensities varied. The grayscale image was first converted into a binary black and white mask image, where cells were shown as white pixels and background in black pixels. The presence of contaminants, such as small hairs, etc, showed up as unusually large blocks of continuous white regions and was identified and excluded from the analysis. The initial scratch proceeded from left to right; however, on occasion, a scratch did not start or end beyond the left and right image borders. To avoid incorporating areas of unscratched, confluent cells, the left and right 25% of the original image were cropped.

An algorithm was implemented using MATLAB 6.5 of Image Processing Toolbox (The MathWorks, Inc., Natick, Mass., USA) to quantify the results. The algorithm calculated the number of white pixels for every row in the image; the resultant curve represented cell density as a function of vertical location. The scratched zone contained significantly less white pixels compared to the rest of the image. Given a hypothetical scratch window, the motility score was defined as: S=AM/AS, in which AS is proportional to the number of cells being removed by the scratch, and AM is proportional to the number of cells moving back into the denuded zone as the result of cell migration. A score close to 1 was assigned to cells with high motility, and a score close to 0 to those with low motility. Since the score was self normalized by cell density, it was comparable across wells and plates.

The vertical center of the scratch may vary from well to well; therefore the algorithm did not assume a fixed scratch location. The above S score was iteratively calculated with every possible scratch center within a given range. Only the minimal possible S score was reported, and the corresponding location is the optimal guess of the scratch center. As input parameters, the method only took the width of the scratch window and a possible range of scratch center. It did not require any training data and was insensitive to variations in cell density. Analysis on some randomly selected wells showed good correlation between the S score and visual inspection.

4. Testing the Assay System with Known Modulators of Cell Migration

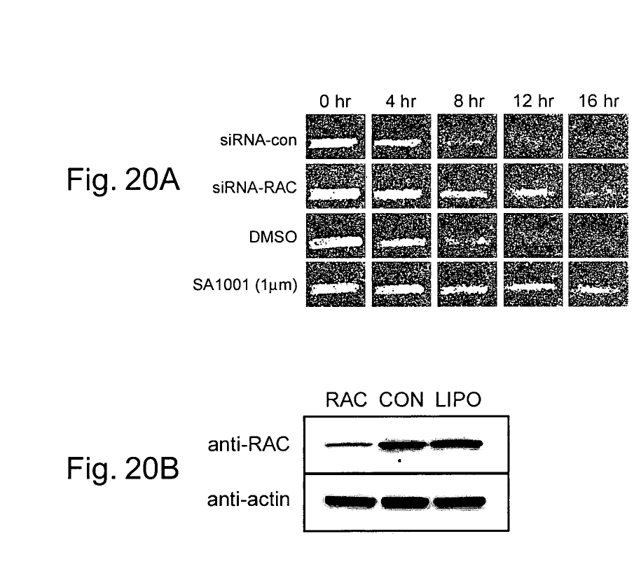

Efficacy of the assay system was first tested by examining migration of a tumor cell line in the presence of known modulators of tumor migration. The temporal migration of SKOV-3 cells, a highly migratory ovarian carcinoma-derived cell line, was monitored in the presence and absence of siRNAs, small molecules and appropriate controls. The efficacy of siRNA-mediated migratory inhibition was assessed using a siRNA against the RhoGTPase Rac1 and compared to a sequence scrambled, FITC-conjugated siRNA control (FIG. 20A). Rac1 is an enzyme which integrates pro-migratory signals with dynamic reorganization of the actin cytoskeleton (Ridely et al. (2003) Science 302:1704-1709, which is incorporated by reference). The assay also included a small molecule, SAI001, which targets the c-Src kinase and its effects were compared to diluent (DMSO) alone. The activated form of c-Src plays a central role in the motility and invasion of cancer cells, including ovarian cancer (Yeatman (2004) Nat. Rev. Cancer 4:470-480 and Wiener et al. (2003) Gynecol Oncol 88:73-79, which are both incorporated by reference). As shown in FIG. 20A, at cell densities ranging from 3,000 to 5,000 cells per well, cells migrated to close the wound typically within 12 hours. In contrast, the addition of Rac siRNA or Src inhibitor significantly inhibited wound closure in the same period of time.

FIG. 20B are photographs of SDS-PAGE/Western blots that demonstrate the knock-down of the Rac1 protein by the Rac1-specific siRNA used in the analysis described with respect to FIG. 20A, compared to a control siRNA (CON) and mock transfected cells (LIPO). Photographs of the same blot re-probed with anti-actin antibody to demonstrate equal loading are also shown FIG. 20B.

In parallel, cell viability was measured in identically treated sister 384-well plates using an ATP-based luminescent assay, to monitor potential toxic effects of siRNA transfection and small molecule inhibition on SKOV-3 cells. The results indicate that in all cases (i.e., the Rac1 and control siRNAs and c-Src inhibition below 3 μM), cell viability was comparable to controls (>90%).

The reproducibility of the assay was tested using a diverse subset of 384 pre-plated siRNAs targeting 192 genes (2 siRNAs per gene plated in duplicate). For these experiments, SKOV-3 cells were reverse transfected on each of three replicate plates, grown to confluency, wounded and incubated for a further 12 hours. Following image capture, wells from each of the three replicate plates were scored by the quantitative algorithm described above and the score from each individual well in each of the three replicate runs was compared to the mean well score using the Pearson correlation coefficient. In each case, r2 was >0.87, demonstrating a high degree of well-to-well consistency.

5. Screening siRNA Library for Pro-Migratory Genes

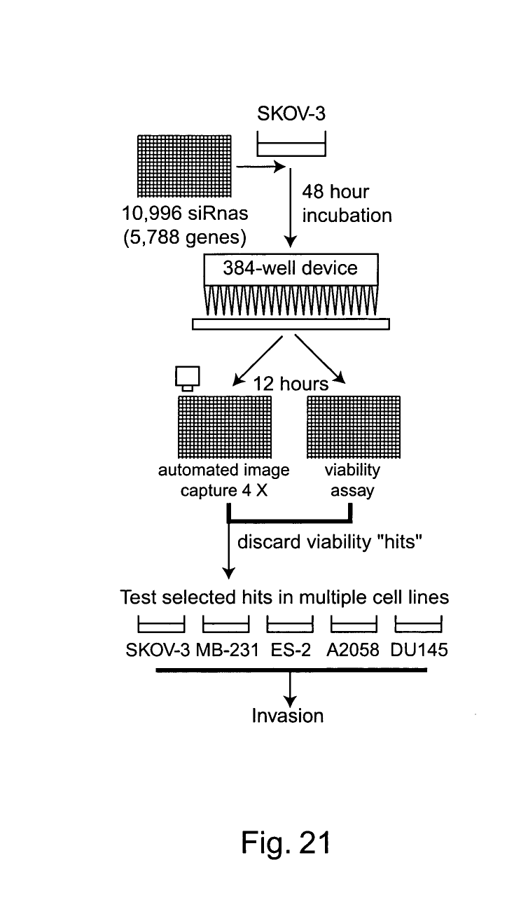

The automated assay system described above was used to screen an siRNA library to identify genes that promote tumor cell motility. The screening employed a pre-plated library of 10,996 siRNAs, targeting 5,234 genes, to identify inhibitors of cellular motility in SKOV-3 cells (FIG. 21). The screen was performed in duplicate (approx. 22,000 wells), as described above, and quantitatively scored. Measurement of cell viability was performed in a set of duplicate siRNA library plates and the luminescence of each well was compared to the normalized mean well intensity of each 384-well plate. Based on measurements from multiple controls that did not affect viability in this assay (i.e., control siRNAs), a cut-off of 0.9 (10% deviation from the plate mean) was adopted, below which siRNAs affecting migration may have resulted from arrested cell growth or cell death and were therefore disregarded.

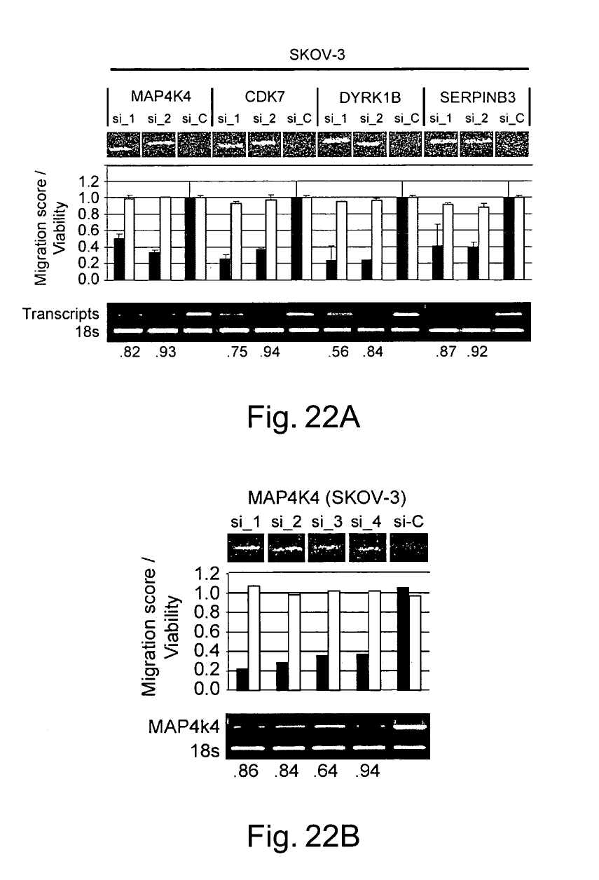

The top 5% of wells in which SKOV-3 cells migrated the least (n=532), were chosen for further analysis, based on a statistical review of the screen. Because of the significant potential for off-target effects when considering the phenotypic effects of single siRNAs, only those transcripts targeted by at least two independent siRNA sequences (n=23) were focused on, with the assumption that a similar phenotypic effect observed with two siRNAs would be less likely to occur by chance. To formally test this assumption, the siRNAs from the library sequences was re-synthesized and transcript knockdown was monitored by semi-quantitative RT-PCR in parallel with migratory inhibition. Of the 48 siRNAs targeting 23 genes, 36 (74%) which target 17 genes yielded migratory phenotypes similar to that of the primary screen. However, the transcripts of only 4 of these 17 genes were significantly diminished by both siRNAs, correlating precisely with the wounding phenotype (FIG. 22). These four genes are MAP4K4 (NM_{—}004834), CDK7 (NM_{—}001799), DYRK1B (NM_{—}004714) and SERPINB3 (NM_{—}006919).

Effect on cell migration by transcriptional inhibition of these 4 genes was further tested in a small series of other migratory carcinoma cells from different anatomic origins, ES-2 (ovarian), MDA-MB-231 (breast), A2058 (melanoma) and DU145 (prostate). This was performed to assess whether the effects of transcriptional inhibition were cell type specific, or reflect more general affects on migration. The results indicate that RNAi-mediated knockdown of MAP4K4 and CDK7 variably affected the migration of all of the cell types tested. In contrast, inhibition of DYRK1B and SerpinB3, affected the motility of SKOV3 and two other cell lines.

B. Example Cellular Motility Assaying System

FIG. 23 is a schematic showing an exemplary system including an information appliance in which various aspects of the present invention may be embodied. As will be understood by practitioners in the art from the teachings provided herein, the invention is optionally implemented in hardware and software. In some embodiments, different aspects of the invention are implemented in either client-side logic or server-side logic. As will also be understood in the art, the invention or components thereof may be embodied in a media program component (e.g., a fixed media component) containing logic instructions and/or data that, when loaded into an appropriately configured computing device, cause that apparatus or system to perform according to the invention. As will additionally be understood in the art, a fixed media containing logic instructions may be delivered to a viewer on a fixed media for physically loading into a viewer's computer or a fixed media containing logic instructions may reside on a remote server that a viewer accesses through a communication medium in order to download a program component.

FIG. 23 shows information appliance or digital device 2300 that may be understood as a logical apparatus (e.g., a computer, etc.) that can read instructions from media 2317 and/or network port 2319, which can optionally be connected to server 2320 having fixed media 2322. Information appliance 2300 can thereafter use those instructions to direct server or client logic, as understood in the art, to embody aspects of the invention. One type of logical apparatus that may embody the invention is a computer system as illustrated in 2300, containing CPU 2307, optional input devices 2309 and 2311, disk drives 2315 and optional monitor 2305. Fixed media 2317, or fixed media 2322 over port 2319, may be used to program such a system and may represent a disk-type optical or magnetic media, magnetic tape, solid state dynamic or static memory, or the like. In specific embodiments, the aspects of the invention may be embodied in whole or in part as software recorded on this fixed media. Communication port 2319 may also be used to initially receive instructions that are used to program such a system and may represent any type of communication connection. Optionally, aspects of the invention are embodied in whole or in part within the circuitry of an application specific integrated circuit (ACIS) or a programmable logic device (PLD). In such a case, aspects of the invention may be embodied in a computer understandable descriptor language, which may be used to create an ASIC, or PLD.

FIG. 23 also includes work perimeter 2327, which includes robotic gripping component 2329, cellular disruption station location 2331 (including cellular disruption component or system 2333), incubation station location 2339 (including incubation component 2341), cell culture plating station location 2343 (including dispensing component 2345), test compound or reagent storage station location 2347 (including test compound or reagent storage component 2349), and assaying component station location 2351 (including assaying component 2353). It will be appreciated that although only a single work perimeter is depicted in FIG. 23, the system components are optionally distributed in more than one work perimeter that each include a robotic gripping component. It will also be appreciated that other components can also be included, such as cell culturing components, etc. These system components are typically operably connected to information appliance 2300 directly or via server 2320.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. An automated cellular disruption system, comprising:
at least one cellular disruption component;
at least one container positioning component structured to position at least one container;
at least one translational mechanism operably connected to the cellular disruption component and/or the container positioning component; and,
at least one controller operably connected to the translational mechanism, which controller is configured to direct the translational mechanism to move the cellular disruption component and/or the container positioning component relative to one another in at least one substantially uniform mode such that the cellular disruption component disrupts cells disposed in the container when the container positioning component positions the container.

2. The automated cellular disruption system of claim 1, wherein the container positioning component comprises a container nest.

3. The automated cellular disruption system of claim 1, wherein the substantially uniform mode comprises one or more selectable parameters selected from the group consisting of: a distance of cellular disruption component and/or container positioning component movement, a pathway of cellular disruption component and/or container positioning component movement, a rate of cellular disruption component and/or container positioning component movement, and a level of force applied by the cellular disruption component and/or the container positioning component on the container.

4. The automated cellular disruption system of claim 1, wherein the translational mechanism comprises at least one linear actuator operably connected to the cellular disruption component.

5. The automated cellular disruption system of claim 1, wherein the translational mechanism comprises at least one air table operably connected to the container positioning component.

6. The automated cellular disruption system of claim 1, wherein the cellular disruption component comprises at least one cellular disruption implement selected from the group consisting of: a radiation source, an electrical source, a thermal source, and a mechanical disruption device.

7. The automated cellular disruption system of claim 6, wherein the mechanical disruption device is selected from the group consisting of: a pipette tip, a prong, a pin, a needle, a scraper, and a razor.

8. The automated cellular disruption system of claim 6, wherein the cellular disruption component comprises a holding block receiving area that comprises a holding block that holds the cellular disruption implement.

9. The automated cellular disruption system of claim 8, wherein the cellular disruption implement comprises at least one locating feature structured to locate the cellular disruption implement relative to the holding block.

10. The automated cellular disruption system of claim 8, wherein the holding block receiving area comprises at least one actuating mechanism operably connected to at least one cellular disruption implement locating component, which actuating mechanism is configured to reversibly move the cellular disruption implement locating component such that the cellular disruption implement locating component applies a substantially constant force to the cellular disruption implement held by the holding block.

11. The automated cellular disruption system of claim 10, wherein an elastomeric material is disposed between the cellular disruption implement locating component and the holding block.

12. The automated cellular disruption system of claim 10, wherein at least one surface of the container positioning component and at least one surface of the cellular disruption implement locating component are substantially parallel with one another.

13. The automated cellular disruption system of claim 10, wherein the cellular disruption implement locating component comprises at least one top support and at least one bottom support operably connected to the actuating mechanism, and wherein the holding block is structured to be positioned between the top and bottom supports.

14. The automated cellular disruption system of claim 13, wherein at least one peg extends from the top support and is resiliently coupled to the top support by a resilient coupling, which peg is configured to contact the cellular disruption implement when the holding block is positioned in the holding block receiving area.

15. The automated cellular disruption system of claim 6, wherein the cellular disruption component comprises multiple cellular disruption implements, and wherein the controller is configured to direct the translational mechanism to move the cellular disruption component and/or the container positioning component relative to one another such that the multiple cellular disruption implements substantially uniformly disrupt the cells disposed in the container when the container positioning component positions the container.

16. The automated cellular disruption system of claim 15, wherein the container positioning component is structured to position a multi-well container, and wherein the multiple cellular disruption implements are configured to correspond to at least a subset of wells of the multi-well container such that the multiple cellular disruption implements substantially uniformly disrupt the cells disposed in the wells of the multi-well container when the translational mechanism moves the cellular disruption component and/or the container positioning component relative to one another and the container positioning component positions the multi-well container.

17. The automated cellular disruption system of claim 6, wherein the cellular disruption component comprises the mechanical disruption device, and wherein the controller is configured to direct the translational mechanism to move the cellular disruption component and/or the container positioning component along a first axis such that the mechanical disruption device deflects away from the first axis upon contacting the container when the container positioning component positions the container.

18. The automated cellular disruption system of claim 17, wherein the controller is configured to direct the translational mechanism to move the cellular disruption component and/or the container positioning component along the first axis such that the mechanical disruption device applies a unit load sufficient to move at least a portion of the container between about 0.20 mm and about 0.55 mm relative to an initial position of the portion of the container.

19. The automated cellular disruption system of claim 17, wherein the controller is configured to direct the translational mechanism to move the cellular disruption component and/or the container positioning component along at least a second axis after the mechanical disruption device contacts the container when the container positioning component positions the container.

20. The automated cellular disruption system of claim 1, comprising one or more additional components selected from the group consisting of:
a robotic gripping component structured to grip and translocate containers between the container positioning component and another location;
an assaying component structured to assay cells;
a material handling component structured to dispense and/or remove material from one or more containers;
an incubation component structured to incubate containers;
a container storage component structured to store containers; and,
a detection component structured to detect detectable signals produced in containers.

21. The automated cellular disruption system of claim 20, wherein the detection component comprises an imaging device that is configured to capture one or more images of cells disposed in the containers.

22. An automated cellular disruption system, comprising:
a cellular disruption component comprising multiple mechanical disruption devices;
a container positioning component structured to position a container;
a translational mechanism operably connected to the cellular disruption component and/or the container positioning component; and,
a controller operably connected to the translational mechanism, which controller is configured to direct the translational mechanism to move the cellular disruption component and/or the container positioning component relative to one another along a first axis such that at least two of the mechanical disruption devices contact at least one surface of the container comprising cells with substantially constant force when the container positioning component positions the container.

23. The automated cellular disruption system of claim 22, wherein the substantially constant force causes the mechanical disruption devices to deflect away from the first axis when the mechanical disruption devices contact the surface of the container.

24. The automated cellular disruption system of claim 22, wherein the mechanical disruption devices are selected from the group consisting of: a pipette tip, a prong, a pin, a needle, a scraper, and a razor.

25. The automated cellular disruption system of claim 22, wherein the container positioning component comprises a container nest.

26. The automated cellular disruption system of claim 22, wherein the container positioning component is structured to position a multi-well container, and wherein the multiple mechanical disruption devices are configured to correspond to at least a subset of wells of the multi-well container such that the multiple mechanical disruption devices contact surfaces of the wells of the multi-well container comprising the cells with the substantially constant force when the container positioning component positions the multi-well container.

27. The automated cellular disruption system of claim 22, wherein the translational mechanism comprises at least one linear actuator operably connected to the cellular disruption component.

28. The automated cellular disruption system of claim 22, wherein the translational mechanism comprises at least one air table operably connected to the container positioning component.

29. The automated cellular disruption system of claim 22, wherein the controller is configured to direct the translational mechanism to move the cellular disruption component and/or the container positioning component along the first axis such that the mechanical disruption devices each apply a unit load sufficient to move at least a portion of the container between about 0.20 mm and about 0.55 mm relative to an initial position of the portion of the container.

30. The automated cellular disruption system of claim 22, wherein the controller is configured to move the cellular disruption component and/or the container positioning component relative to one another along at least a second axis when the container positioning component positions the container and the mechanical disruption devices are in contact with the surface of the container.

31. The automated cellular disruption system of claim 22, comprising one or more additional components selected from the group consisting of:
a robotic gripping component structured to grip and translocate containers between the container positioning component and another location;
an assaying component structured to assay cells;
a material handling component structured to dispense and/or remove material from one or more containers;
an incubation component structured to incubate containers;
a container storage component structured to store containers; and,
a detection component structured to detect detectable signals produced in containers.

32. The automated cellular disruption system of claim 31, wherein the detection component comprises an imaging device that is configured to capture one or more images cells disposed in the containers.

33. The automated cellular disruption system of claim 22, wherein the cellular disruption component comprises a holding block receiving area that comprises a holding block that holds the mechanical disruption devices.

34. The automated cellular disruption system of claim 33, wherein the mechanical disruption devices each comprise at least one locating feature that is structured to locate the mechanical disruption devices relative to the holding block.

35. The automated cellular disruption system of claim 33, wherein the holding block receiving area comprises at least one actuating mechanism operably connected to at least one cellular disruption implement locating component, which actuating mechanism is configured to reversibly move the cellular disruption implement locating component such that the cellular disruption implement locating component applies a substantially constant force to the mechanical disruption devices held by the holding block.

36. The automated cellular disruption system of claim 35, wherein an elastomeric material is disposed between the cellular disruption implement locating component and the holding block.

37. The automated cellular disruption system of claim 35, wherein at least one surface of the container positioning component and at least one surface of the cellular disruption implement locating component are substantially parallel with one another.

38. The automated cellular disruption system of claim 35, wherein the cellular disruption implement locating component comprises at least one top support and at least one bottom support operably connected to the actuating mechanism, and wherein the holding block is structured to be positioned between the top and bottom supports.

39. The automated cellular disruption system of claim 38, wherein at least one peg extends from the top support and is resiliently coupled to the top support by a resilient coupling, which peg is configured to contact the cellular disruption implement when the holding block is positioned in the holding block receiving area.

40. An automated cellular disruption system, comprising:
at least one cellular disruption component comprising a holding block receiving area that is structured to receive a holding block that is structured to hold at least one cellular disruption implement;
at least one translational mechanism operably connected to the cellular disruption component; and,
at least one controller operably connected to the translational mechanism, which controller is configured to direct the translational mechanism to move the cellular disruption component such that the cellular disruption component disrupts cells disposed in at least one container when the holding block holds the cellular disruption implement, the holding block receiving area receives the holding block, and the container is positioned relative to the cellular disruption component.

41. The automated cellular disruption system of claim 40, wherein the translational mechanism comprises a linear actuator operably connected to the cellular disruption component.

42. The automated cellular disruption system of claim 40, wherein the controller is configured to direct the translational mechanism to move the cellular disruption component in at least one substantially uniform mode.

43. The automated cellular disruption system of claim 40, comprising at least one container positioning component structured to position one or more containers.

44. The automated cellular disruption system of claim 43, wherein the container positioning component comprises a container nest.

45. The automated cellular disruption system of claim 43, the translational mechanism is operably connected to the container positioning component and the controller is configured to move the container positioning component and the cellular disruption component relative to one another.

46. The automated cellular disruption system of claim 45, wherein the translational mechanism comprises at least one air table operably connected to the container positioning component.

47. The automated cellular disruption system of claim 40, comprising one or more additional components selected from the group consisting of:
a robotic gripping component structured to grip and translocate containers between the container positioning component and another location;
an assaying component structured to assay cells;
a material handling component structured to dispense and/or remove material from one or more containers;
an incubation component structured to incubate containers;
a container storage component structured to store containers; and,
a detection component structured to detect detectable signals produced in containers.

48. The automated cellular disruption system of claim 47, wherein the detection component comprises an imaging device that is configured to capture one or more images cells disposed in the containers.

49. The automated cellular disruption system of claim 40, comprising the holding block.

50. The automated cellular disruption system of claim 49, wherein the holding block holds the cellular disruption implement.

51. The automated cellular disruption system of claim 50, wherein the cellular disruption implement comprises a mechanical disruption device, and wherein the controller is configured to direct the translational mechanism to move the cellular disruption component along a first axis such that the mechanical disruption device deflects away from the first axis upon contacting the container when the container is positioned relative to the cellular disruption component.

52. The automated cellular disruption system of claim 51, wherein the controller is configured to direct the translational mechanism to move the cellular disruption component along the first axis such that the mechanical disruption device applies a unit load sufficient to move at least a portion of the container between about 0.20 mm and about 0.55 mm relative to an initial position of the portion of the container.

53. The automated cellular disruption system of claim 51, wherein the controller is configured to direct the translational mechanism to move the cellular disruption component along at least a second axis after the mechanical disruption device contacts the container when the container is positioned relative to the cellular disruption component.

54. The automated cellular disruption system of claim 50, wherein the cellular disruption implement is selected from the group consisting of: a radiation source, an electrical source, a thermal source, and a mechanical disruption device.

55. The automated cellular disruption system of claim 54, wherein the cellular disruption implement comprises at least one locating feature.

56. The automated cellular disruption system of claim 54, wherein the mechanical disruption device is selected from the group consisting of: a pipette tip, a prong, a pin, a needle, a scraper, and a razor.

57. The automated cellular disruption system of claim 50, wherein the holding block holds multiple cellular disruption implements, and wherein the controller is configured to direct the translational mechanism to move the cellular disruption component such that the cellular disruption implements substantially uniformly disrupt the cells disposed in the container when the container is positioned relative to the cellular disruption component.

58. The automated cellular disruption system of claim 57, wherein the multiple cellular disruption implements are configured to correspond to at least a subset of wells of a multi-well container such that the cellular disruption implements substantially uniformly disrupt the cells disposed in the wells of the multi-well container when the multi-well container is positioned relative to the cellular disruption component.
